(19) United States
(12) Patent Application Publication
(10) Pub. No.: US 2009/0130776 A1
(43) Pub. Date: May 21, 2009

(54) BINDING PROTEIN MOLECULE

(75) Inventors: Takeshi Imamura, Chigasaki-shi (JP); Hidenori Shiotsuka, Kawasaki-shi (JP); Kazuhiro Ban, Tokyo (JP); Takahisa Ibii, Yokohama-shi (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/995,911

(22) PCT Filed: Sep. 1, 2006
(86) PCT No.: PCT/JP2006/317808

§ 371 Date: Jan. 16, 2008

(30) Foreign Application Priority Data
Sep. 1, 2005 (JP) 2005-253337

(51) Int. Cl. G01N 33/543; C07K 16/18; B01J 19/00

(52) U.S. Cl. 436/518; 530/387.1; 422/68.1

(57) ABSTRACT

A binding protein molecule, characterized in that it has a first domain having a binding site to an inhibitor of non-specific adsorption in which the domain comprises a part of the variable region of an antibody as the binding site and a second domain having a binding site to a target substance in which the domain comprises a part of the variable region of an antibody as the binding site, wherein the first and second domains are bound via a linker.

### TECHNICAL FIELD

The present invention relates to a binding protein molecule comprising more than one domains and having a binding site to a target substance.

### BACKGROUND ART

Up to the present, immunoassays using a protein, particularly an antibody or a fragment thereof containing its molecular recognition site have been taken advantage of in the field of medical diagnosis. Recent progress in peripheral techniques such as microdetection and microstructure fabrication have led to the development of various devices to detect and quantify analytes (target substances) through application of antigen-antibody reactions. For instance, devices for diagnosis, detection and/or screening such as immunosensors and protein microarrays have been now used. Additionally, applications of these devices based on genomic analysis to detection and search of antigenic proteins associated with certain diseases, and detection of certain pathogenic microorganisms or viruses have become broader. Furthermore, these devices have begun to find a wider application, such as detection of environmental hormones or allergenic substances in the field of environmental measurement or process management of food production, and detection of toxic chemicals even in the military field.

However, the amounts of samples obtained for use in various detections or searches as described above are limited, and target substances are often contained only in a minute amount among many substances which are present together in a complex manner. Accordingly, they require a highly precise (reproducible), sensitive analytical method. The analytical method must also provide highly reliable analysis results without mistake.

In order to establish an analytical method with high levels of precision, sensitivity and reliability, optimization of antigen-antibody reactions or similar protein-specific recognition reactions, which plays an central role in the relevant technology, is necessary in addition to a further progress in the peripheral techniques described above. In such reactions, some problems remaining to be solved satisfactorily include:

(1) fixation of orientation of molecules to capture target substances such as antibodies; and
(2) nonspecific adsorption of contaminants other than target substances to reactive regions.

A variety of techniques described below has been developed to solve these problems.

Japanese Patent No. 3007423 discloses an immunosorbent material comprising a specific binding agent immobilized on a porous solid-phase support material. In this immunosorbent material, the specific binding agent comprises at least one variable region protein (VH and/or VL) which is not bound to any other substantial portion of one or more original antibodies. The publication discloses a method of immobilizing a small specific binding substance such as Fv directly on a solid phase without a significant loss of its activity. The publication also discloses a method of immobilizing a specific binding agent as antibody fragment on a solid phase support where the binding agent comprises components described in i) and ii) as follows:

i) at least one variable region protein (VH and/or VL) which is not bound to any other substantial portion of one or more original antibodies; and
ii) a chemical group (preferably a peptidyl group) which makes no contribution to the basic specific binding property, but may be coupled to the solid-phase support material by chemical or any other means without a significant effect on the basic specific binding activity of the binding agent.

Japanese Patent No. 3095415 discloses a soluble immunocomplex comprising (a) an antibody fragment, and also (b) a loaded support or (c) a non-antibody moiety, and retaining the immunoreactivity of the antibody fragment, where (a), (b) and (c) are described as follows:

(a) a glycosylated antibody fragment selected from the group consisting of Fab, Fab′, F(ab)₂, F(ab′)2, Fv and a single-chain Fv each of which comprises a light-chain variable region having an artificial carbohydrate portion bound around position 18 of its amino acid sequence;
(b) a loaded support comprising a polymer support having at least one free amine group and also comprising more than one unit of one selected from the group consisting of a drug, a toxin, a chelating agent, a boron addend and a detectable labeled molecule, which is covalently bonded to the polymer support, wherein the loaded support is covalently bonded to the carbohydrate portion of the antibody fragment through the at least one free amine group of the polymer support; and
(c) at least one non-antibody moiety selected from the group consisting of a drug, a toxin, a chelating agent, a boron addend and a detectable labeled molecule, which is covalently bonded to the carbohydrate portion of the antibody fragment.

Japanese Patent Application Laid-Open No. 2002-520618 discloses a protein array comprising patches. The patches comprise:

(a) a substrate;
(b) at least one organic thin film present on some portions of a surface of the substrate or on the entire surface; and
(c) a plurality of patches disposed on known separate areas on the organic thin film coated on the portions of the substrate surface.
Each of the patches comprises a protein immobilized on the underlying organic thin film. Further, the monolayer contained by the organic thin film comprises a self-assembled monolayer comprising a molecular species expressed by the formula:

(X)aR(Y)b Formula

wherein R is a spacer, X is a functional group binding R to the surface, Y is a functional group binding the protein to the monolayer, and a and b each independently represent an integer.

It has been required, as described above, to establish an analytic methodology with higher levels of precision, sensitivity and reliability in devices for diagnosis, detection or screening as various devices based on molecular recognition for proteins, above all, detection of antigen-antibody reactions, have been available more widely. Such devices include immunoassay kits, immunosensors and protein microarrays. To deal with these requirements, research and development through various approaches are under progress to solve relevant problems, especially those represented by:

(1) fixation of orientation of molecules to capture target substances such as antibodies; and
(2) nonspecific adsorption of contaminants other than target substances to reactive regions.
However, the techniques relating to the devices are not necessarily sufficient to solve the two problems at the same time. Therefore, a widely applicable, highly reliable means for solving them is sought to develop an element with higher levels of sensitivity and precision.

### DISCLOSURE OF THE INVENTION

The binding protein molecule provided by the present invention is characterized in that it has at least one component selected from a first group consisting of components (1) and (4) and at least one component selected from a second group consisting of components (2) and (3), wherein components (1) to (4) are described as follows:

(1) A domain having a binding site to an inhibitor of non-specific adsorption, wherein the domain comprises at least a part of the variable region of an antibody heavy chain,
(2) A domain having a binding site to a target substance, wherein the domain comprises at least a part of the variable region of an antibody light chain,
(3) A domain having a binding site to the target substance, wherein the domain comprises at least a part of the variable region of an antibody heavy chain and
(4) A domain having a binding site to an inhibitor of non-specific adsorption, wherein the domain comprises at least a part of the variable region of an antibody light chain.

Further, the binding protein molecule of the present invention is characterized in that it has a first domain having a binding site to the inhibitor of non-specific adsorption in which the domain comprises a part of the variable region of an antibody as the binding site and a second domain having a binding site to the target substance in which the domain comprises a part of the variable region of an antibody as the binding site, wherein the first and second domains are bound via a linker.

The protein complex of the present invention is a protein complex having the protein molecule with a binding site to the target substance and the inhibitor of non-specific adsorption bound to the protein molecule, characterized in that the protein molecule with a binding site to the target substance is a binding protein molecule having the above constitution.

The substrate-protein complex of the present invention is a substrate-protein complex having a substrate, the inhibitor of non-specific adsorption disposed on at least a portion on the substrate and the binding protein molecule with a binding site to the target substance, characterized in that the binding protein molecule with a binding site to the target substance is a protein complex having the above constitution.

The method of producing the substrate-protein complex of the present invention is a method of producing the substrate-protein complex having the substrate and the protein with a binding site to the target substance, characterized in that it has the steps of:

(step A) disposing the inhibitor of non-specific adsorption on the surface of the substrate and

(step B) binding the inhibitor of non-specific adsorption disposed on the substrate with the protein with a binding site to the target substance,

wherein the protein with a binding site to the target substance is a binding protein molecule having the above constitution.

The target substance capturing element of the present invention is a target substance capturing element having the substrate and a target substance capturing protein immobilized to the surface of the substrate, in which the region where the target substance capturing protein is immobilized to is the reactive region, characterized in that the target substance capturing protein is a protein molecule having the above constitution and the protein molecule is immobilized to the substrate surface via the inhibitor of non-specific adsorption disposed on the reactive region.

The target substance detection method of the present invention is characterized in that it has the step of contacting a target substance capturing element having the above constitution with a sample and the step of detecting the state in which the target substance is captured by the target substance capturing element when the sample comprises the target substance.

The detection device for the target substance of the present invention is characterized in that it has a reactive region for reacting a target substance capturing element having the above constitution and the sample and a detection means for detecting the state in which the target substance is captured by the target substance capturing element.

The method of capturing the target substance of the present invention is characterized in that it has the step of contacting a target substance capturing element having the above constitution with the sample to capture the target substance with the target substance capturing element and the step of washing the target substance capturing element with which the target substance was captured to separate the captured target substance from the substance that was not captured by the target substance capturing element.

The capturing device for the target substance of the present invention is characterized in that it has a reactive region for reacting a target substance capturing element having the above constitution and the sample and means for washing and removing the target substance captured by the target substance capturing element and the substance that was not captured by the target substance capturing element from the target substance capturing element.

In the present invention, an antibody or an antibody fragment that has affinity towards an inhibitor of non-specific adsorption and an antibody or an antibody fragment that has affinity towards a target substance are used in combination to constitute a binding protein molecule. As a result, in the reactive region on the element, various non-specific bindings and adsorptions other than the specific binding of the target substance to the target substance capture molecule which consists of binding protein molecule can be effectively prevented. In addition to the above constitution, in the present invention, the constitution of the binding protein molecule and the complex is formed with the inhibitor of non-specific adsorption adsorbed to the substrate surface of the reactive region.

The following effects can be achieved concurrently by these constitutions.

(1) The antibody fragment of the binding protein molecule having affinity to the inhibitor of non-specific adsorption is directed towards the substrate via the inhibitor of non-specific adsorption, and consequently the antibody fragment having affinity to the target substance is directed elsewhere, other than the substrate. As a result, the molecule can be directed with sufficient utilization of the target substance recognition region.

(2) Except for the region occupied by the binding protein molecule, the substrate surface located on the reactive region is occupied by the inhibitor of non-specific adsorption that prevents non-specific adsorption of impurities other than the target substance to the reactive region. As a result, non-specific adsorption of impurities other than the target substance to the reactive region is prevented.

According to the present invention which has such effects, a separation/analysis technique with higher accuracy, sensitivity and reliability can be established using a device for molecular recognition of proteins, in particular for diagnosis, detection or screening based on antigen-antibody reaction. Examples of these devices include an immunoassay kit, an immunosensor, and a protein microarray.

### BRIEF DESCRIPTION OF THE DRAWINGS

FIG. 1 illustrates an example of constitutions presented by the binding protein molecule of the present invention;

FIG. 2 illustrates an example of constitutions presented by the binding protein molecule of the present invention;

FIG. 3 illustrates an example of constitutions presented by the binding protein molecule of the present invention;

FIG. 4 illustrates an example of constitutions presented by the binding protein molecule of the present invention;

FIG. 5 illustrates an example of constitutions presented by the binding protein molecule of the present invention;

FIG. 6 illustrates an example of constitutions presented by the binding protein molecule of the present invention;

FIG. 7 illustrates an example of constitutions presented by the binding protein molecule of the present invention;

FIG. 8 illustrates an example of constitutions presented by the protein complex of the present invention;

FIG. 9 illustrates an example of constitutions presented by the protein complex of the present invention;

FIG. 10 illustrates an example of constitutions presented by the protein complex of the present invention;

FIG. 11 illustrates an example of constitutions presented by the protein complex of the present invention;

FIG. 12 illustrates an example of constitutions presented by the protein complex of the present invention;

FIG. 13 illustrates an example of constitutions presented by the protein complex of the present invention;

FIG. 14 illustrates an example of constitutions presented by the protein complex of the present invention;

FIG. 15 illustrates an example of constitutions presented by the protein complex of the present invention;

FIG. 16 illustrates an example of constitutions presented by the protein complex of the present invention;

FIG. 17 illustrates an example of constitutions presented by the protein complex of the present invention;

FIG. 18 illustrates an example of constitutions presented by the protein complex of the present invention;

FIG. 19 illustrates an example of constitutions presented by the protein complex of the present invention;

FIG. 20 illustrates an example of constitutions presented by the protein complex of the present invention;

FIG. 21 illustrates an example of constitutions presented by the protein complex of the present invention;

FIG. 22 illustrates an example of constitutions presented by the substrate-protein complex of the present invention;

FIG. 23 illustrates an example of constitutions presented by the substrate-protein complex of the present invention;

FIG. 24 illustrates an example of constitutions presented by the substrate-protein complex of the present invention;

FIG. 25 illustrates an example of constitutions presented by the substrate-protein complex of the present invention;

FIG. 26 illustrates an example of constitutions presented by the substrate-protein complex of the present invention;

FIG. 27 illustrates an example of constitutions presented by the substrate-protein complex of the present invention;

FIG. 28 illustrates an example of constitutions presented by the substrate-protein complex of the present invention;

FIG. 29 illustrates an example of constitutions presented by the substrate-protein complex of the present invention;

FIG. 30 illustrates an example of constitutions presented by the substrate-protein complex of the present invention;

FIG. 31 illustrates an example of constitutions presented by the substrate-protein complex of the present invention;

FIG. 32 illustrates an example of constitutions presented by the substrate-protein complex of the present invention;

FIG. 33 illustrates an example of constitutions presented by the substrate-protein complex of the present invention;

FIG. 34 illustrates an example of constitutions presented by the substrate-protein complex of the present invention;

FIG. 35 illustrates an example of constitutions presented by the substrate-protein complex of the present invention;

FIG. 36 illustrates an example of processes for producing the substrate-protein complex of the present invention;

FIG. 37 illustrates an example of processes for producing the substrate-protein complex of the present invention;

FIG. 38 illustrates capturing a target substance with the substrate-protein complex of the present invention;

FIG. 39 illustrates the structure of a substrate having a flow channel formed therein;

FIG. 40 illustrates the structure of pUT-XX2;

FIG. 41 illustrates a process flow to make a vector (pPEG-HEL) for expressing VH(PEG)-VL(HEL); and

FIG. 42 illustrates a process flow to make a vector (pHEL-PEG) for expressing VH(HEL)-VL(PEG).

### BEST MODE FOR CARRYING OUT THE INVENTION

First of all, the following terms will be described which are inhibitor of nonspecific adsorption, antibody, immunoglobulin, antibody fragment, variable domain, light chain/heavy chain, functional portion and binding protein molecule.

### <Inhibitor of Nonspecific Adsorption>

The inhibitor of nonspecific adsorption may not be limited if it is useful to attain the desired effect of the present invention. It has preferably a function to suppress or prevent the nonspecific adsorption of contaminants other than target substances to reactive regions which is, above all, one of the problems to be solved by the present invention. Such an inhibitor of nonspecific adsorption may be either a biological material or a non-biological material. Known inhibitors of nonspecific adsorption of biological origin include biological materials making no interaction with target substances, such as bovine serum albumin, casein and skim milk. Further, derivatives of these biological materials produced by modification thereof are available if they function as inhibitor of nonspecific adsorption.

Furthermore, non-biological polymers having at least one functional group capable of inhibiting nonspecific adsorption located at least partially thereon are available as inhibitor of nonspecific adsorption. Such functional groups may be of any type if they can prevent or suppress nonspecific adsorption of substances to the substrate surface. The functional groups may include a hydroxyl group, a methoxy group, an ethoxy group, a propoxy group, an isopropoxy group, a butyl group, a t-butyl group, 2-hydroxyethyl group, 2-hydroxypropyl group, 3-hydroxypropyl group, 2-hydroxyisopropyl group, 2-hydroxybutyl group, 3-hydroxybutyl group, 4-hydroxybutyl group, a styrenyl group, a sulfonyl group, a phosphonyl group, an amino group, a methylamino group, an ethylamino group, an isopropylamino group, an amide group, a methylamide group, an ethylamide group, an isopropylamide group, a pyrrolidonyl group, an ethylene glycol group, a phospholipid group, a choline group, a phosphatidyl choline group and derivatives thereof. The functional groups may be used to bind a hydrophilic molecule such as sugars later. Polymers capable of inhibiting nonspecific adsorption are preferred that partially contain at least one selected from the group consisting of an ethylene glycol group and its derivative groups, among those groups. Also, polymers capable of inhibiting nonspecific adsorption are preferred that partially contain at least one selected from the group consisting of a phosphatidyl choline group and its derivative groups.

These substances as inhibitor of nonspecific adsorption may be disposed on the surface of a substrate by known methods. For instance, an inhibitor of nonspecific adsorption may be disposed on the substrate surface by immersing the substrate in a solution containing the inhibitor of nonspecific adsorption for a certain time. The inhibitor of nonspecific adsorption may be disposed on the substrate surface by modifying the substrate surface and forming a chemical bond between the inhibitor of nonspecific adsorption and the substrate surface. The methods of modifying the substrate include, but not limited to, formation of a hydrophobic coating thereon and formation of a self-assembled monolayer (SAM) on the substrate with an alkanethiol derivative.

### <Antibody or Immunoglobulin>

The term “an antibody” means an immunoglobulin either produced in living organisms in nature or synthesized totally or partially via genetic modification, protein engineering, or even organic reactions or the like. Every derivative of the immunoglobulin still capable of specific binding is also a member of “antibodies”. The term also includes any protein either homologous to the binding domain of an immunoglobulin or having a binding domain highly homologous to that domain (including chimeric antibodies and humanized antibodies). Such “antibodies” or “immunoglobulins” used here may be produced in living organisms in nature, synthesized totally or partially via genetic engineering or the like, or additionally modified.

The “antibody” or “immunoglobulin” may be a monoclonal antibody or a polyclonal antibody.

The “antibody” or “immunoglobulin” may be a member of any class of immunoglobulin, including any class of human immunoglobulin (IgG, IgM, IgA, IgD or IgE). However, those derived from the IgG class are preferred in the present invention.

### <Antibody Fragment>

The term “an antibody fragment” refers to any molecular species or complex contained in an antibody which constitutes a part of the full-length antibody or immunoglobulin. The antibody fragment should preferably retain at least a major part of the specific binding capacity of the full-length antibody. Examples of the antibody fragment include, but not limited to, the fragments Fab, Fab′, F(ab′)2, scFv, Fv, diabody and Fd.

The antibody fragment may be produced by any means. For instance, the antibody fragment may be produced by enzymatic or chemical fragmentation of an intact antibody. Alternatively, it may be produced by recombination of a gene encoding a partial antibody sequence. Alternatively, the antibody fragment may be produced by total or partial synthesis. The antibody fragment may be a single-chain antibody fragment if necessary. Alternatively, the fragment may comprise multiple chains linked by, for example, a disulfide (—S—S—) linkage. The fragment may be also a complex composed of multiple molecules if necessary. A functional antibody fragment typically includes at least about 50 amino acids, and more typically at least about 200 amino acids.

### <Variable Domain and Light Chain/Heavy Chain>

The term “a variable domain” refers to a domain at the forefront of an immunoglobulin which has an amino acid sequence portion variable with an individual target substance (antigen) to exhibit a specific binding/capturing function depending on the type of the antigen, and is usually represented by Fv. The Fv consists of “a variable domain in the heavy chain (may be described as VH hereinafter)” and “a variable domain in the light chain (may be described as VL hereinafter)”, and an immunoglobulin G typically contains two VH domains and two VL domains.

### <Functional Portion>

The term “a functional portion in the heavy-chain or light-chain variable domain of an immunoglobulin (may be simply described as a functional portion hereinafter)” refers to a portion within the variable region which is actually responsible for its specificity to a target substance (antigen). In a narrower sense, it refers to a portion called a CDR (a complementarity determining region, also called a hypervariable region) plus its peripheral portion, and in a broader sense also refers to these portions plus a framework structure which is required by them to make the antigen-antibody combination and contributes to the CDR structure.

### <Binding Protein Molecule>

The binding protein molecule in the present invention may be composed of an “antibody” or “immunoglobulin”, or an “antibody fragment” or “antibody fragment”, as described above. Also, it may be composed of variable regions (Fv) in the antibody or a part thereof, for example, a heavy-chain variable region (VH) or a light-chain variable region (VL) each of which is a constituent of the Fv, or a part thereof.

The binding protein molecule in the present invention is a binding protein molecule comprising at least one selected from the first group consisting of components (1) and (4) and at least one selected from the second group consisting of components (2) and (3), wherein components (1) to (4) are described as follows:

(1) a domain having a binding site to an inhibitor of non-specific adsorption and comprising at least a part of the variable region of the antibody heavy chain;
(2) a domain having a binding site to a target substance and comprising at least a part of the variable region of the antibody light chain;
(3) a domain having a binding site to the target substance and comprising at least a part of the variable region of the antibody heavy chain; and
(4) a domain having a binding site to the inhibitor of non-specific adsorption and comprising at least a part of the variable region of the antibody light chain.

It should be noted that the target substance bound to the domain in (2) and the target substance bound to the domain in (3) represent the same molecule.

More desirably, the binding protein molecule has a linked body where two components are linked together among the components described above. For instance, if the binding protein molecule is composed of three domains, it may contain a linked body where two of the three domains are linked together. If the binding protein molecule is composed of four domains, it may also contain a linked body where two of the four domains are linked together, or two linked bodies where two of the four domains per linked body are linked together. Still more desirably, the two linked bodies are formed by combination of components (1) and (2) as well as components (3) and (4). In other words, it is still more desirable that the binding protein molecule comprises at least the first linked body consisting of components (1) and (2) and the second linked body consisting of components (3) and (4).

The antibody fragment having a binding site to an inhibitor of non-specific adsorption is exemplified by domains having affinity to polyethylene glycol as will be described below. The domain of component (1) preferably comprises at least one of the amino acid sequences given by SEQ ID NOS: 1 to 3 shown as:

SEQ ID NO: 1: NYWIN;
SEQ ID NO: 2: NSYPGSSSTNYNEKFK;
and
SEQ ID NO: 3: YCARSG.

Further, the domain of component (1) preferably has at least one selected from the group consisting of the amino acid sequences given by (A) to (F) shown as:

(A) the amino acid sequence given by SEQ ID NO: 1 (NYWIN);
(B) the amino acid sequence given by SEQ ID NO: 2 (NSYPGSSSTNYNEKFK);
(C) the amino acid sequence given by SEQ ID NO: 3 (YCARSG);
(D) an amino acid sequence formed by providing deletion, substitution or addition of one or more amino acids into the amino acid sequence given by SEQ ID NO: 1;
(E) an amino acid sequence formed by providing deletion, substitution or addition of one or more amino acids into the amino acid sequence given by SEQ ID NO: 2; and
(F) an amino acid sequence formed by providing deletion, substitution or addition of one or more amino acids into the amino acid sequence given by SEQ ID NO: 3.

The domain of component (4) preferably comprises at least one of the amino acid sequences given by SEQ ID NOS: 4 to 6 shown as:

the amino acid sequence given by SEQ ID NO: 4
(RSSQSIVHSNGNTYLD);
the amino acid sequence given by SEQ ID NO: 5
(KVSNRFS);
and
the amino acid sequence given by SEQ ID NO: 6
(FQGSHVPLT).

The domain of component (4) preferably has at least one selected from the group consisting of the amino acid sequences given by (A) to (F) shown as:

(A) the amino acid sequence given by SEQ ID NO: 4 (RSSQSIVHSNGNTYLD);
(B) the amino acid sequence given by SEQ ID NO: 5 (KVSNRFS);
(C) the amino acid sequence given by SEQ ID NO: 6 (FQGSHVPLT);
(D) an amino acid sequence formed by providing deletion, substitution or addition of one or more amino acids into the amino acid sequence given by SEQ ID NO: 4;
(E) an amino acid sequence formed by providing deletion, substitution or addition of one or more amino acids into the amino acid sequence given by SEQ ID NO: 5; and
(F) an amino acid sequence formed by providing deletion, substitution or addition of one or more amino acids into the amino acid sequence given by SEQ ID NO: 6.

In addition, an example of a domain having affinity to an inhibitor of non-specific adsorption is a domain having affinity to phosphatidyl choline as described below.

The domain of component (1) preferably comprises at least one of the amino acid sequences given by SEQ ID NOS: 7 to 9 shown as:

SEQ ID NO: 7: GFTFSDFYME;
SEQ ID NO: 8: ASRNKANDYTTEYSASVK;
and
SEQ ID NO: 9: DYYGSSYWYFDV.

The domain of component (1) preferably has at least one selected from the group consisting of the amino acid sequences given by (A) to (F) shown as:

(A) the amino acid sequence given by SEQ ID NO: 7 (GFTFSDFYME);
(B) the amino acid sequence given by SEQ ID NO: 8 (ASRNKANDYTTEYSASVK);
(C) the amino acid sequence given by SEQ ID NO: 9 (DYYGSSYWYFDV);
(D) an amino acid sequence formed by providing deletion, substitution or addition of one or more amino acids into the amino acid sequence given by SEQ ID NO: 7;
(E) an amino acid sequence formed by providing deletion, substitution or addition of one or more amino acids into the amino acid sequence given by SEQ ID NO: 8; and
(F) an amino acid sequence formed by providing deletion, substitution or addition of one or more amino acids into the amino acid sequence given by SEQ ID NO: 9.

Also, the domain of component (4) preferably comprises at least one of the amino acid sequences given by SEQ ID NOS: 10 to 12 shown as:

SEQ ID NO: 10: TASESLYSSKHVHYLA;
SEQ ID NO: 11: GASNRYI;
and
SEQ ID NO: 12: AQFYSYPL.

The domain of component (4) preferably has at least one selected from the group consisting of the amino acid sequences given by (A) to (F) shown as:

(A) the amino acid sequence given by SEQ ID NO: 10 (TASESLYSSKHVHYLA);
(B) the amino acid sequence given by SEQ ID NO: 11 (GASNRYI);
(C) the amino acid sequence given by SEQ ID NO: 12 (AQFYSYPL);
(D) an amino acid sequence formed by providing deletion, substitution or addition of one or more amino acids into the amino acid sequence given by SEQ ID NO: 10;
(E) an amino acid sequence formed by providing deletion, substitution or addition of one or more amino acids into the amino acid sequence given by SEQ ID NO: 11; and
(F) an amino acid sequence formed by providing deletion, substitution or addition of one or more amino acids into the amino acid sequence given by SEQ ID NO: 12.

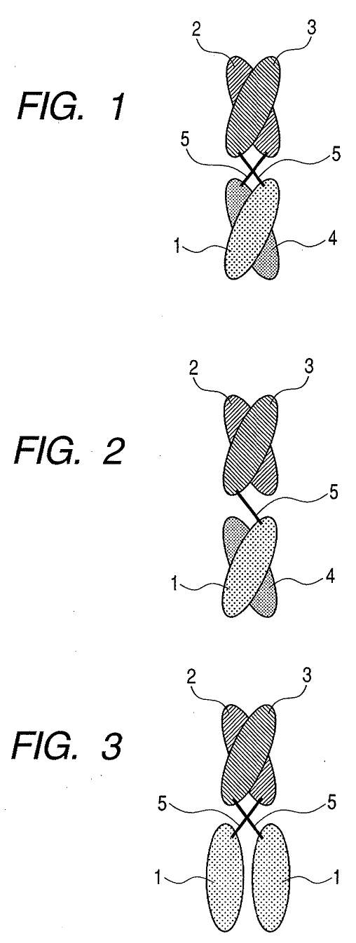

An example of the binding protein molecule of the present invention using such an antibody or antibody fragment will be described in detail below. FIG. 1 shows a typical diagram of the inventive binding protein molecule in the most preferable form. One of domains 1 and 4 has a variable region in the antibody heavy chain (VH) and a binding site to the inhibitor of non-specific adsorption comprising a functional portion of the variable region. The other of the domains has a variable region in the antibody light chain (VL) and a binding site to the inhibitor of non-specific adsorption comprising a functional portion of the variable region. Accordingly, the combination of domains 1 and 4 has binding sites to the inhibitor of non-specific adsorption and forms an Fv molecule. Furthermore, one of domains 2 and 3 has a variable region in the antibody heavy chain (VH) and a binding site to the target substance comprising a functional portion of the variable region. The other of the domains has a variable region in the antibody light chain (VL) and a binding site to the target substance comprising a functional portion of the variable region. Accordingly, the combination of domains 2 and 3 has binding sites to the target substance and forms an Fv molecule. The target substance will be described later.

Domains 1 and 3 as well as domains 4 and 2 are linked together through a linker 5, respectively. The linker 5 has a peptide structure with about 1 to 10 amino acid residues, more preferably about 1 to 5 amino acid residues. When the combination VL-VH are made so that domains 2 and 3 as well as domains 1 and 4 have a complementary correlation, respectively, only one linker 5 to link together domains 1 and 3 may be used as shown in FIG. 2.

Also, when the combination VL-VH are made so that domains 2 and 3 as well as domains 1 and 4 have a complementary correlation, respectively, only one linker to link together domains 2 and 4 may be used rather than it is used to link together domains 1 and 3 as shown in FIG. 2.

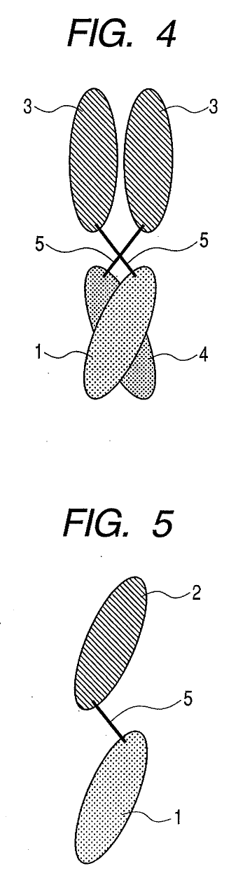

In addition, the inventive binding protein molecule may not necessarily constitute an Fv fragment. For instance, it may be constituted of the combinations of domains 1 and 2 and domains 1 and 3 as shown in FIG. 3, or of the combinations of domains 3 and 1 and domains 3 and 4 as shown in FIG. 4. In the constitution shown in FIG. 3, domain 1 may be replaced by domain 4, and in the constitution shown in FIG. 4, domain 3 may be replaced by domain 2. To summarize, the protein molecule may be only constituted so as to exhibit a more desirable specificity and/or affinity to the target substance or inhibitor of non-specific adsorption.

FIG. 5 shows a typical diagram of the smallest constitution presented by the inventive binding protein molecule. The numbering corresponds to that in FIG. 1. A domain comprising at least a part of a variable region in the antibody light chain (VL) or heavy chain (VH) has CDR (also called a hypervariable region, meaning a portion actually having a specific affinity to a substance of interest, i.e., a binding site) at three different locations. Therefore, if for the target substance and the inhibitor of non-specific adsorption each one domain has a good affinity to them, the domains may be selected from the first and second groups, respectively, which were described previously, to make a constitution having them linked by a linker. Paying attention to FIG. 5, the inventive binding protein molecule may be considered as a binding protein molecule comprising a first domain and a second domain and having the first domain and the second domain linked together through a linker, wherein the first domain has a binding site to an inhibitor of non-specific adsorption and comprises a part of an antibody variable region as the binding site and the second domain has a binding site to a target substance and comprises a part of an antibody variable region as the binding site.

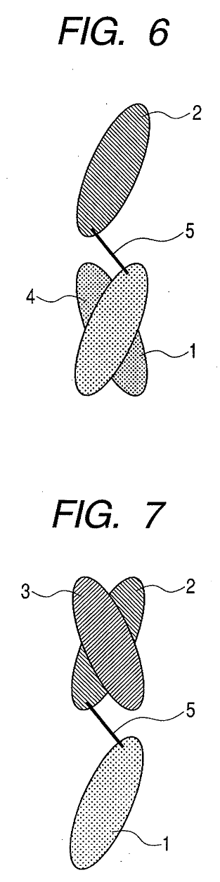

FIG. 6 shows a typical diagram of another constitution presented by the inventive binding protein molecule. The numbering corresponds to that in FIG. 1. The binding protein molecule shown in FIG. 6 has a three-domain structure. In the three-domain structure, a single domain comprising at least a part of a variable region in the antibody light chain (VL) or heavy chain (VH) has a good affinity to the target substance for itself. In contrast, a domain comprising at least a part of a variable region in the antibody light chain (VL) and another domain comprising at least a part of a variable region in the heavy chain (VH) have an affinity in combination to the inhibitor of non-specific adsorption.

In this way, the number of domains constituting the binding protein molecule may be determined depending on the purpose, where it consists of the number of domains (at least one) necessary to acquire an affinity to the target substance and the number of domains (at least one) necessary to acquire an affinity to the inhibitor of non-specific adsorption.

FIG. 7 shows a typical diagram of another constitution presented by the inventive binding protein molecule. The numbering corresponds to that in FIG. 1. In the form of the three-domain constitution, a single domain comprising at least a part of a variable region in the antibody light chain (VL) or heavy chain (VH) has a good affinity to the inhibitor of non-specific adsorption on its own. In contrast, a domain comprising at least a part of a variable region in the antibody light chain (VL) and another domain comprising at least a part of a variable region in the heavy chain (VH) have an affinity in combination to the target substance.

The most preferable form (FIG. 1) of the inventive binding protein molecule is what is called a “diabody” (Hollinger et al., Proc. Natl. Acad. Sci. USA 90, 6444-6448, 1993). The “diabody” has antibody variable VH and VL domains linked together by a very short linker. The linker is designed so as to be too short to form a single-chain Fv fragment by bonding a VH domain to a VL domain, and consequently the VH and VL domains each forming one of two chains associate with each other to form a dimer. As a result, a molecule with two binding sites meaning a “diabody” is produced (Perisic et al., Structure 2, 1217-1226, 1994). A bispecific “diabody” is produced via expression of double-chain structures of VH(A)-VL(B) and VH(B)-VL(A) in a cell. Herein, VL means a variable (V) region in the antibody light (L) chain and VH means a variable (V) region in the heavy (H) chain, and these variable regions are bound to the antigen (A) or (B). Association of the VH and VL portions forms a heterodimeric fragment having functionally effective binding sites.

In FIG. 1, domain 2 and domain 3 have a binding site to a target substance. Domain 2 comprises at least a part of the antibody light chain and domain 3 comprises at least a part of a heavy-chain variable region. The domains used here may be those derived from known gene sequences.

### <Methods of Obtaining Binding Protein Molecule>

An antibody heavy-chain variable region (VH) or antibody light-chain variable region (VL) capable of binding to a target substance, which is used in the inventive binding protein molecule, may be obtained by one method of immunization. Specifically, an animal is immunized with an inhibitor of non-specific adsorption or a target substance described above, and treated to collect an antibody (a polyclonal antibody) according to the standard procedure from blood serum, milk (normal milk or initial milk), egg yolk or the like, depending on the species of the animal, and the antibody may be further treated, e.g., fragmented as necessary to provide a desirable variable region.

On the other hand, the desirable variable region may be also obtained by cell fusion using a monoclonal-antibody producing cell. In this process, an antibody producing cell such as a spleen cell or a lymph node cell is first isolated to provide a monoclonal-antibody producing cell. The monoclonal-antibody producing cell thus obtained is fused with a myeloma cell to make the resulting hybridoma proliferate semipermanently. The myeloma cell used for cell fusion may include a cell line such as Sp2/0-Ag14 (SP2). Then, the resulting hybridomas are screened to select a hybridoma producing a desired monoclonal antibody, which is then cultured to collect the antibody.

Further, the inventive binding protein molecule can be obtained by known techniques, such as production of a quadroma, that is, a further fusion of hybridomas disclosed in U.S. Pat. No. 4,474,493. Furthermore, the inventive binding protein molecule can be also obtained by chemical recombination of half molecules [Science 229: 81 (1985)]. Immunization, fusion, hybridoma selection, antibody collection, etc. can be carried out according to known standard procedures. A gene encoding VH or VL can be obtained using known primers capable of amplifying the VH or VL antibody gene.

Additionally, a process of constructing a VH or VL gene library, expressing fusion proteins derived therefrom on the surface of a virus or cell in an exhaustive manner, and screening them for binding to an inhibitor of non-specific adsorption or a target substance is available.

The gene library can be obtained, for example, from umbilical cord blood, tonsil, bone marrow, or peripheral blood cells, spleen cells or the like. Specifically, first of all, mRNA is extracted from human peripheral blood cells and used to synthesize cDNA. Then, a sequence encoding human VH or VL is used as a probe to construct a cDNA library of the human VH or VL. Primers capable of greatly amplifying, for example, each of the human VH families (VH 1 to 7) and primers capable of amplifying the human VL are known. Primers are combined for each VH as well as for the VL, and used to carry out RT-PCR and obtain genes encoding the VH and the VL. Based on the antibody sequence determined, a gene encoding the hypervariable region can be synthesized artificially to construct a diversified, large-scale library and make it available.

The phage display method is also available. In the phage display method, a gene library encoding VH, VL or a complex comprising them (e.g., Fab or scFv) is first ligated to a gene encoding a phage envelope protein to construct a phagemid library. Then, it is transfected into E. coli to transform the microorganism, where phages having different VHs or VLs as a part of the envelope protein are developed. In the same way as described above, a desired VH or VL can be selected from the phages with respect to binding to an inhibitor of non-specific adsorption or a target substance. Genes encoding VHs or VLs presented as fusion protein to the phage are present in phagemids within the phage, and therefore DNA sequencing of those is available as one of the methods to identify fusion proteins.

The obtainment of VH or VL having a specific affinity to phosphatidyl choline will be exemplified below using phosphatidyl choline as inhibitor of non-specific adsorption by means of the phage display method.

Construction of a phage display library and in vitro screening may be conducted by known methods. First of all, a known commercial phage library is provided, or a phage library is constructed from a gene library constructed as described above so that antibody genes described above may be expressed on the envelope of the phagemid vector. Thereafter, phage clones presenting binding antibody fragments to phosphatidyl choline are screened. The layer of phosphatidyl choline is formed on titer plate wells or on a solid phase such as an insoluble support. Then, a solution of the phage library is added on the phosphatidyl choline monolayer and incubated for a proper period of time. Unbound phages are washed away and phages left on the solid phase are recovered. E. coli is infected with the recovered phages to construct a new phage library. This series of operations (a round) provides the concentration of the ligand-presenting phages with a significant affinity to the target to a factor of 10² to 10⁴. When biopanning is repeated where the host E. coli is infected with recovered phages to amplify them, a phage library is obtained after 3-5 rounds that contains only phages presenting sequences with a significant affinity to the target. DNA present in the phages thus selected is extracted from them and determined for its nucleotide sequence to obtain VH or VL expressed by the phages and capable of specifically binding to phosphatidyl choline.

### <Method of Producing Binding Protein Molecule>

The binding protein molecule according to the present invention can be produced by incorporating a gene encoding each required domain into a vector, such as a plasmid or cosmid, and introducing the vector into a cell such as E. coli or yeast to express the domain in the cell. In the production, the signal peptide may be incorporated into the vector to induce secretion of the binding protein molecule out of the cell before isolation and purification. If it is produced as inclusion body in the cell, the inclusion body may be denatured with a denaturing agent such as urea or guanidin-HCl, and subjected to gradient dialysis or loaded with a molecular chaperon or the like to renature its conformation (refolding). Furthermore, the binding protein molecule may be produced in a “cell-free in vitro system” in the presence of a cell extract from E. coli, wheat germ, or even insect, animal or similar cells.

### <Protein Complex>

The protein complex according to the present invention is a complex where a binding protein molecule constituted as described above is constituted so as to have an inhibitor of non-specific adsorption bound thereto, through a binding site of the binding protein molecule to the inhibitor of non-specific adsorption. FIG. 8 shows a typical diagram of the most preferred form of the inventive protein complex. One of domains 1 and 4 has a binding site to the inhibitor of non-specific adsorption and consists of a variable region (VH) in the antibody heavy chain and a functional portion thereof. The other of the domains has a binding site to the inhibitor of non-specific adsorption and consists of a variable region (VL) in the antibody light chain and a functional portion thereof. Accordingly, the combination of domains 1 and 4 has binding sites to the inhibitor of non-specific adsorption and forms an Fv molecule. Furthermore, one of domains 2 and 3 has a binding site to the target substance (the target substance will be described later) and comprises a variable region (VH) in the antibody heavy chain and a functional portion thereof. The other of the domains has a binding site to the target substance and comprises a variable region (VL) in the antibody light chain and a functional portion thereof. Accordingly, the combination of domains 2 and 3 has binding sites to the target substance and forms an Fv molecule. The binding site present in at least one of domains 1 and 4 is bound to the inhibitor of non-specific adsorption 6. Domains 1 and 3 as well as domains 4 and 3 are linked together through a linker 5, respectively. The linker 5 has a peptide structure with about 1 to 10 amino acid residues, more preferably about 1 to 5 amino acid residues.

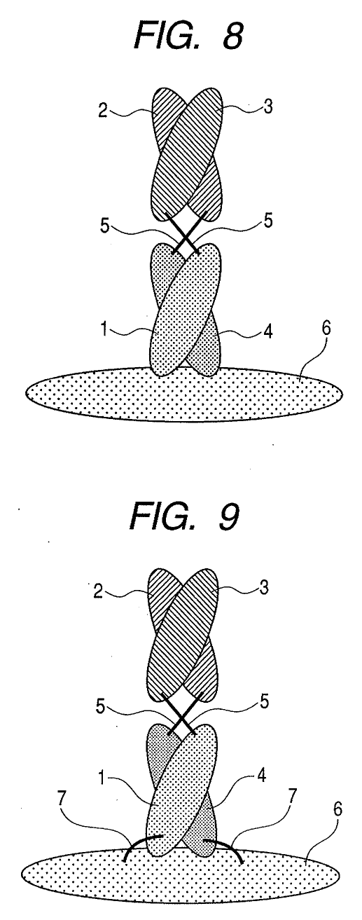

If it is intended to reinforce the bonding between domains 1, 4, which have each a binding site to the inhibitor of non-specific adsorption, and the inhibitor of non-specific adsorption, it can be attained by forming a covalent bond between the domains and the inhibitor via a bridging molecule shown in FIG. 9. The bridging molecule will be described later.

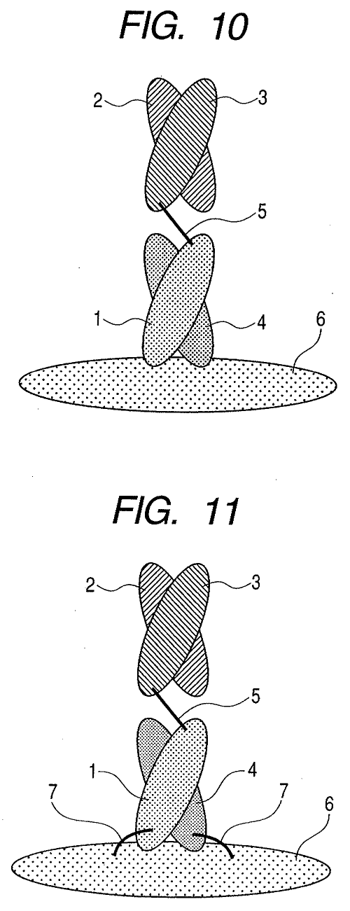

When the combination VL-VH are made so that domains 2 and 3 as well as domains 1 and 4 have a complementary correlation, respectively, only one linker 5 to link together domains 1 and 3 may be used as shown in FIGS. 10 and 11. In the constitution shown in FIG. 10 or 11, only one linker 5 to link together domains 2 and 4 may be used instead.

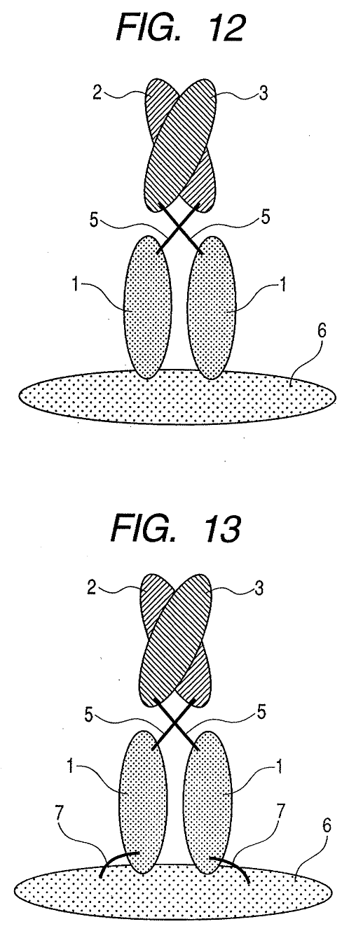

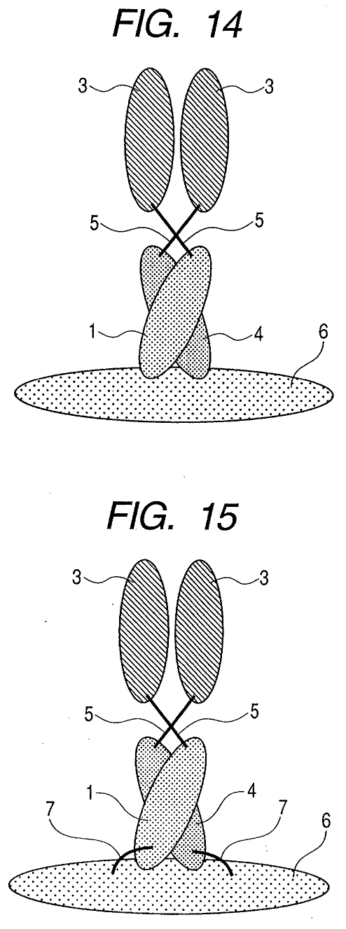

In addition, the inventive protein complex may not necessarily constitute an Fv fragment. For instance, it may be constituted of the combinations of domains 1 and 2 and domains 1 and 3 as shown in FIGS. 12 and 13. Alternatively, it may be also constituted of the combinations of domains 3 and 1 and domains 3 and 4 as shown in FIGS. 14 and 15. In the constitution shown in FIGS. 12 and 13, domain 1 may be replaced by domain 4, and in the constitution shown in FIGS. 14 and 15, domain 3 may be replaced by domain 2. To summarize, the protein complex may be only constituted so as to exhibit a more desirable specificity and/or affinity to the target substance or inhibitor of non-specific adsorption.

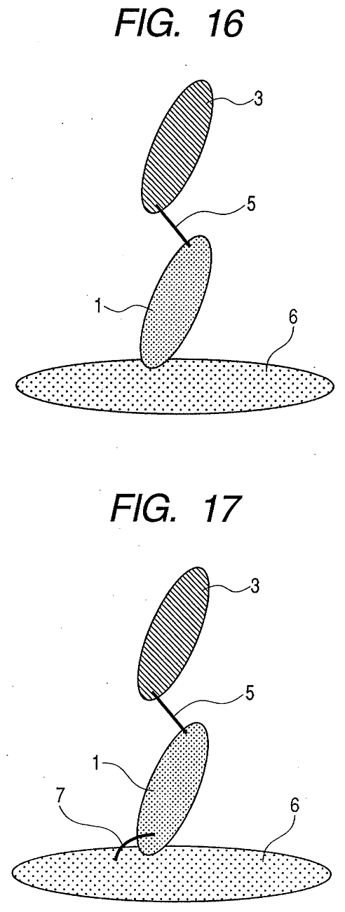

FIGS. 16 and 17 show a typical diagram of the smallest constitution presented by the inventive protein complex. The numbering corresponds to that in FIG. 8. A domain comprising at least a part of a variable region in the antibody light chain (VL) or heavy chain (VH) has CDR (also called a hypervariable region, meaning a portion actually having a specific affinity to a substance of interest, i.e., a binding site) at three different locations. Therefore, if for the target substance and the inhibitor of non-specific adsorption each one domain has a good affinity to them, the domains may be selected from the first and second groups, respectively, which were described previously, so that the binding protein molecule contained in this protein complex has a constitution having the domains linked by a linker.

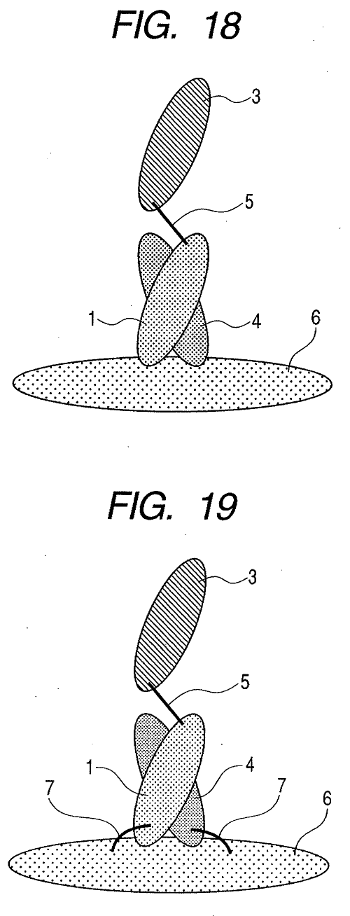

FIGS. 18 and 19 show a typical diagram of another constitution presented by the inventive protein complex. The numbering corresponds to that in FIG. 8. In this three-domain structure, a single domain comprising at least a part of a variable region in the antibody light chain (VL) or heavy chain (VH) has a good affinity to the target substance for itself. In contrast, a domain comprising at least a part of a variable region in the antibody light chain (VL) and another domain comprising at least a part of a variable region in the heavy chain (VH) have an affinity in combination to the inhibitor of non-specific adsorption. The inventive binding protein molecule can form a protein complex by making the molecule into a three-domain constitution and binding at least one of the domains 1 and 4 to the inhibitor of non-specific adsorption.

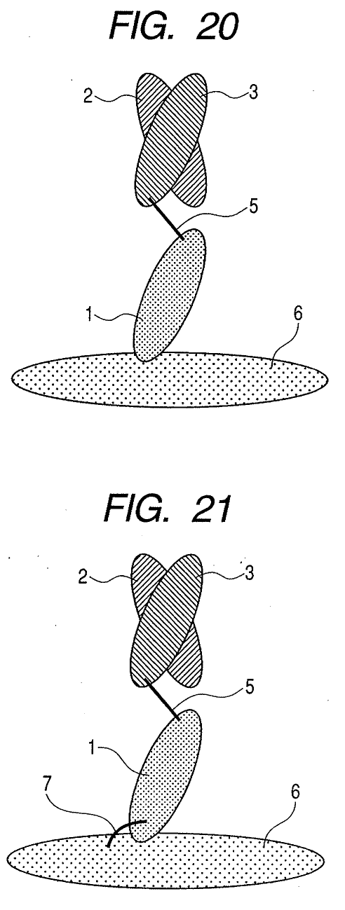

FIGS. 20 and 21 show a typical diagram of another constitution presented by the inventive protein complex. The numbering corresponds to that in FIG. 8. In this three-domain structure, a single domain comprising at least a part of a variable region in the antibody light chain (VL) or heavy chain (VH) has a good affinity to the inhibitor of non-specific adsorption for itself. In contrast, a domain comprising at least a part of a variable region in the antibody light chain (VL) and another domain comprising at least a part of a variable region in the heavy chain (VH) have an affinity in combination to the target substance. The inventive binding protein molecule can form a protein complex by making the molecule into a three-domain constitution and binding at least one of the domains 1 and 4 to the inhibitor of non-specific adsorption.

If it is intended to reinforce the bonding between domains 1 and/or 4, which have each an affinity to the portion 6 composed of the inhibitor of non-specific adsorption, and the portion 6 composed of the inhibitor of non-specific adsorption, it can be attained by forming a covalent bond between the domains and the portion via a bridging molecule shown in FIG. 23. The bridging molecule will be described later.

### <Substrate-Protein Complex>

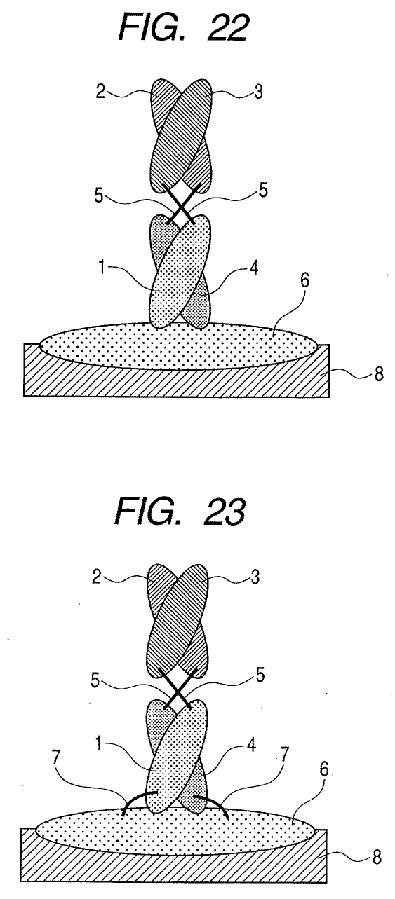

A substrate-protein complex according to the present invention takes a constitution having a protein complex with the above constitution immobilized on a surface of a substrate. FIG. 22 shows a typical diagram of the most preferred form of the inventive substrate-protein complex. The structure of the substrate-protein complex having domains 1 to 4 is also illustrated in FIG. 8. In this substrate-protein complex, the portion 6 formed of the inhibitor of non-specific adsorption from the protein complex is disposed on the surface of the substrate 8 by physical and/or chemical attachment thereto. The substrate will be described later.

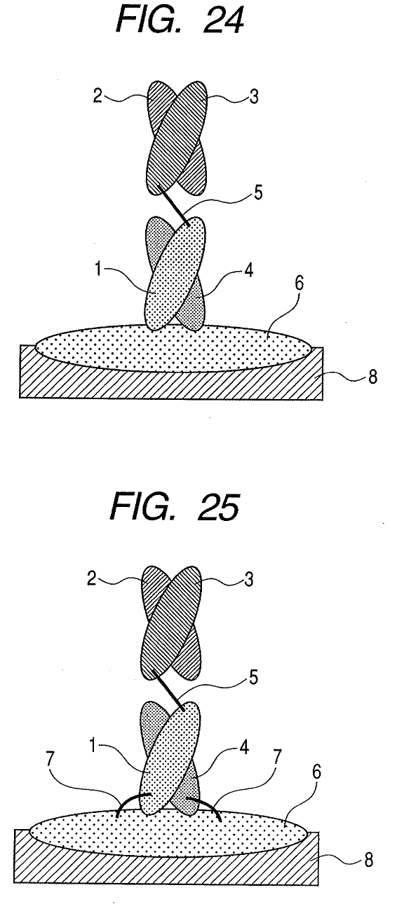

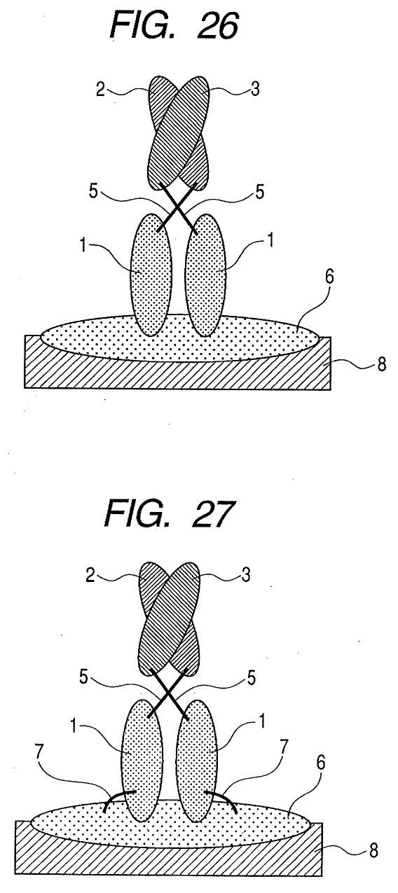

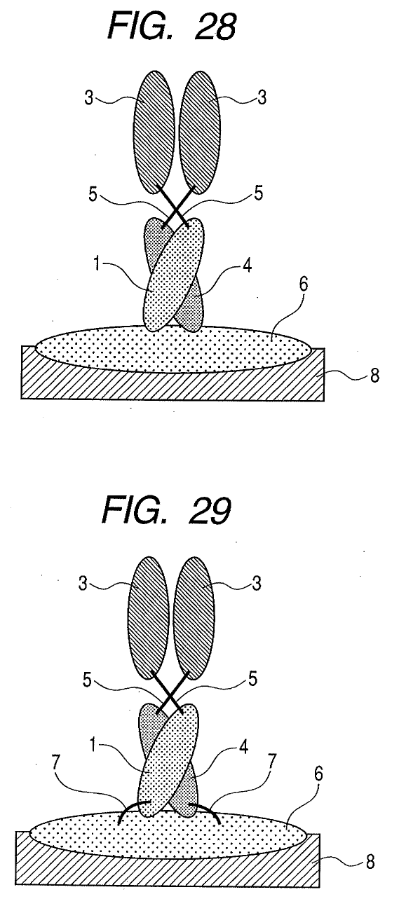

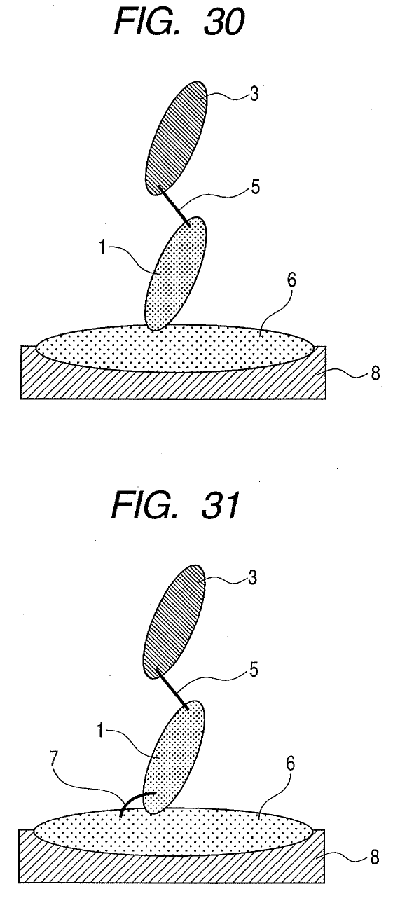

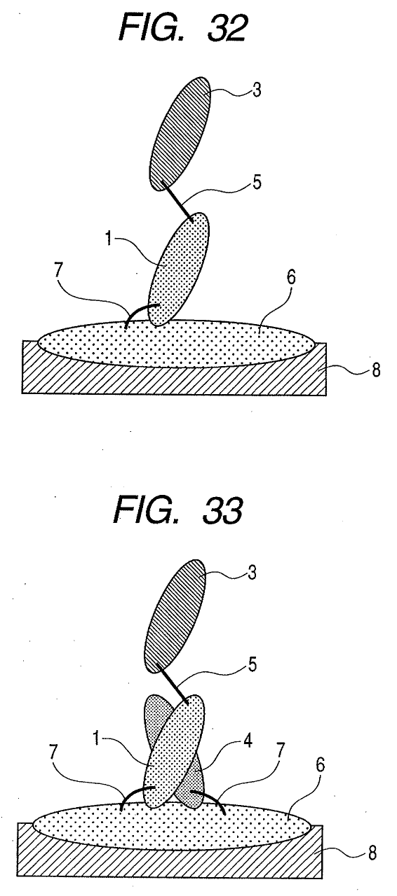

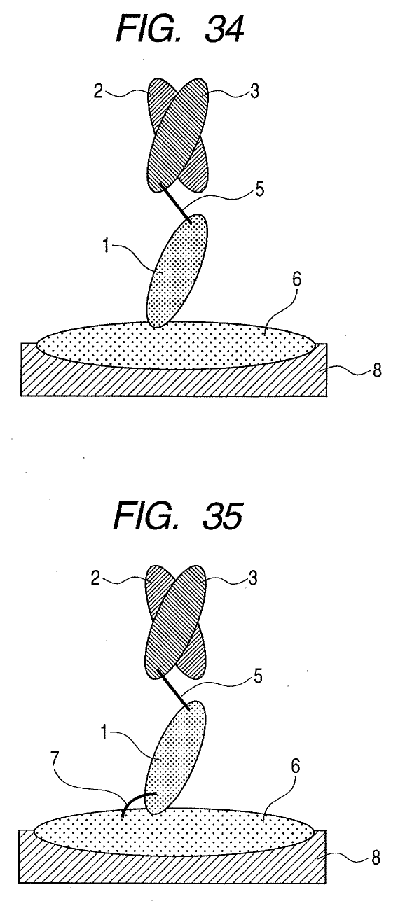

The binding protein molecule constituting the inventive substrate-protein complex may further take various forms as described previously referring to FIGS. 2 to 7 (i.e., the forms shown in FIGS. 24, 26, 28, 30, 32 and 34). Furthermore, bonding between domains 1, 4 and the portion formed of the inhibitor of non-specific adsorption can be reinforced in the same various constitutions (FIGS. 25, 27, 29, 31, 33 and 35).

The inventive substrate-protein complex may be produced by a process comprising:

step A which is a step of exposing an inhibitor of non-specific adsorption to a substrate to adsorb the inhibitor of non-specific adsorption to a surface of the substrate; and
step B which is a step of exposing and binding a binding protein molecule to the inhibitor of non-specific adsorption.

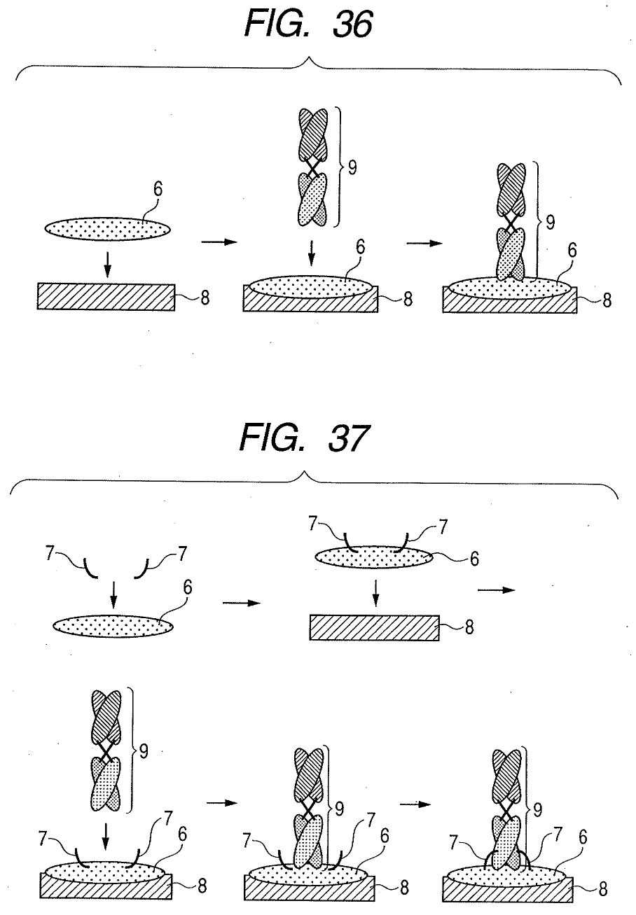

FIG. 36 shows a typical diagram of a process of producing a substrate-protein complex where a binding protein molecule comprises four domains (1) to (4). In the diagram, reference numeral 8 denotes a substrate, reference numeral 6 denotes an inhibitor of non-specific adsorption, and reference numeral 9 denotes a binding protein molecule.

The process of producing a substrate-protein complex according to the present invention may comprise, in addition to steps A and B, a step (step C) of forming a covalent bond between the inhibitor of non-specific adsorption and the binding protein molecule via a bridging molecule. Here, FIG. 37 shows a typical diagram of a process of producing a substrate-protein complex where the binding protein molecule comprises four domains (1) to (4). In this process, the inhibitor of non-specific adsorption 6 undergoes a reaction with one of the two active groups of the bridging molecule 7 and then a contact with the substrate 8 to immobilize the inhibitor of non-specific adsorption 6 on the substrate surface. Then, the binding protein molecule 9 is exposed and bound to the inhibitor of non-specific adsorption and then bonded to the inhibitor of non-specific adsorption 6 by the other active group of the bridging molecule 7.

In addition, the binding protein molecule 9 may be preliminarily bridged with the inhibitor of non-specific adsorption by the bridging molecule 7 to form the protein complex, which may be then immobilized in the substrate. Generally, the inhibitor of non-specific adsorption has a higher affinity to the substrate, and therefore the moiety consisting of the inhibitor of non-specific adsorption may be disposed on the substrate with a better chance. In order to control the density of the protein complex on the substrate here, a solution containing only the protein complex may be used to immobilize it on the substrate.

The inhibitor of non-specific adsorption constituting the inventive protein complex may be additionally constituted to form a stronger bond with a domain of the first group, as described previously, present in the binding protein. It will be described in detail later in the section of “covalent bond and photobridging molecule”.

### <Substrate>

The substrate may be formed of any material in any form if it can produce a substrate-protein complex according to the present invention. The material of the substrate may be of any type if it can produce the inventive substrate-protein complex, and selected from metals, metal oxides, inorganic semiconductors, organic semiconductors, glasses, ceramics, organic polymers (natural polymers or synthetic polymers), plastics or the likes. The substrate may have a portion formed of a composite material comprising two or more of these materials. The shape of the substrate used in the invention may be in any shape if it can produce the inventive substrate-protein complex, and selected from the group consisting of the forms of plate, granule, porous body, projection, fiber, cylinder and mesh. The substrate may have a combination of the forms as necessary.

The organic polymer used for the substrate may include an organic polymer produced by polymerization of a polymerizable monomer selected from the group consisting of styrenic polymerizable monomers such as styrene, α-methylstyrene, β-methylstyrene, o-methylstyrene, m-methylstyrene, p-methylstyrene, 2,4-dimethylstyrene, p-n-butylstyrene, p-tert-butylstyrene, p-n-hexylstyrene, p-n-octylstyrene, p-n-nonylstyrene, p-n-decylstyrene, p-n-dodecylstyrene, p-methoxystyrene and p-phenylstyrene; acrylic polymerizable monomers such as methyl acrylate, ethyl acrylate, n-propyl acrylate, iso-propyl acrylate, n-butyl acrylate, iso-butyl acrylate, tert-butyl acrylate, n-amyl acrylate, n-hexyl acrylate, 2-ethylhexyl acrylate, n-octyl acrylate, n-nonyl acrylate, cyclohexyl acrylate, benzyl acrylate, dimethylphosphate-ethyl acrylate, diethylphosphate-ethyl acrylate, dibutylphosphate-ethyl acrylate and 2-benzoyloxyethyl acrylate; methacrylic polymerizable monomers such as methyl methacrylate, ethyl methacrylate, n-propyl methacrylate, iso-propyl methacrylate, n-butyl methacrylate, iso-butyl methacrylate, tert-butyl methacrylate, n-amyl methacrylate, n-hexyl methacrylate, n-2-ethylhexyl methacrylate, n-octyl methacrylate, n-nonyl methacrylate, diethylphosphate-ethyl methacrylate and dibutylphosphate-ethyl methacrylate; and vinylic polymerizable monomers including: methylenealiphatic monocarboxylate esters; vinyl esters such as vinyl acetate, vinyl propionate, vinyl benzoate, vinyl butyrate, vinyl benzoate and vinyl formate; vinyl ethers such as vinyl methyl ether, vinyl ethyl ether and vinyl isobutyl ether; and vinyl ketones such as vinyl methyl ketone, vinyl hexyl ketone and vinyl isopropyl ketone.

Examples of inorganic solids include, but are not limited to, clay minerals such as kaolinite, bentonite, talc and mica; metal oxides such as silica, alumina, titanium dioxide, zinc oxide, magnetite, ferrite, NbTa oxide composite, WO₃, In₂O₃, MoO₃, V₂O₅ and SnO₂; insoluble inorganic salts such as silica gel, hydroxyapatite and calcium phosphate gel; metals such as gold, silver, platinum and copper; semiconductor compounds such as GaAs, GaP, ZnS, CdS and CdSe, glass, quartz glass and silicon, or complexes thereof can be used.

Particular examples of materials for the substrate according to the shape of substrate include the following:

Examples of a film include films consisting of plastics such as polyethylene terephthalate (PET), diacetate, tricetate, cellophane, celluloid, polycarbonate, polyimide, polyvinyl chloride, polyvinylidene chloride, polyacrylate, polyethylene, polypropylene and polyester.

Examples of a porous polymeric film include porous polymeric films consisting of polyvinyl chloride, polyvinyl alcohol, acetyl cellulose, polycarbonate, nylon, polypropylene, polyethylene, teflon etc.

Examples of a fabric for a substrate in a film or sheet form include wooden board, glass plate, silicon substrate, fabrics such as cotton, rayon, acryl, silk, polyester, etc. Examples of a paper in a film or sheet form include wood-free paper, wood-containing paper, art paper, bond paper, recycled paper, baryta paper, cast coated paper, corrugated paper, resin coated paper etc. that can be used in a film or sheet form. These materials in a film or sheet form may be smooth or may be uneven.

Examples of a substrate consisting of a composite material include a substrate such as silicon, silica, glass and quartz glass on which microchannels and holes (pores) are provided by techniques such as photolithography, etching and sandblasting, or those on which a thin film of gold, silver or platinum is provided on the surface thereof.

Examples of other forms of the substrate include substrates such as PDMS (polydimethylsiloxane), PMMA (polymethyl methacrylate), PET (polyethylene terephthalate), PC (polycarbonate), PS (polystyrene) on which microchannels and holes (pores) are provided.

Further examples of other forms of the substrate include, but are not limited to, carbon nanotube, carbon nanohorn, fullerene diamond or aggregates thereof; a nanowhisker consisting of for example alumina, carbon, fullerene and ZnO; a mesoporous thin film, microparticle and monolith structure consisting of for example SiO₂, aluminosilicate, other metallosilicates, TiO₂, SnO₂ and Ta₂O₅; microparticles such as gold, silver, copper and platinum; iron oxide microparticles such as magnetite, ferrite, hematite, γ-hematite and maghemite; aluminum silicon mixed film and its anodized silicon oxide nanostructure; porous alumina thin film; alumina nanohole structure; and silicon nanowire.

### <Covalent Bond and Photobridging Molecule>

In the present invention, it is desirable to utilize a bridging molecule having active functional groups that enable covalent bonding with the protein at two or more positions in the molecule, to allow for the binding of the inhibitor of non-specific adsorption and the domain selected from the first group. As such active functional groups, compounds having a chemical bridging molecule species that is an ordinary protein bridging or a modified reactive group, for example, N-hydroxysuccinimide ester, maleimido, carbodiimide, adipimidate, pimelimidate, suberimidate, picolinimidate, hydrazide, pyridyldithio, glycidyl, epoxy and aldehyde groups can be utilized.

In addition, examples of photobridging molecule species include molecular species such as azide, diazo, diazirine and carbonyl. These molecular species become excited species by radiation of light, for example, azide becomes nitrene, diazo and diazirine becomes carbene and carbonyl becomes excited carbonyl or carbonyl radical, and each of these nucleophilically reacts with a proximal protein molecule. Molecular scaffolds and functional groups which comprise these molecular species and are preferred from the practical standpoint are phenyl azide group, trifluoromethyl diazirine group or benzophenone group. Among these, particularly a phenyl diazirine atomic group having a trifluoromethyl group as shown in chemical formula (1) below is capable of efficiently progressing in bridging reaction in a light irradiation condition that has little effect on biomolecules (wavelength: approximately 350 nm, irradiation time: approximately 1 hour).

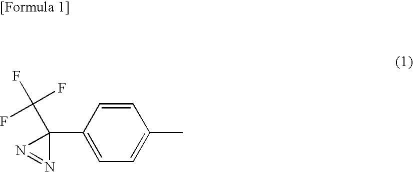

This is described in detail in Current Opinion in Medicinal Chemistry, 2, 271-288 (2002).

A bridging molecule comprising a photoreactive atomic group having two or more molecular species as a combination of these chemical bridging molecule species and photobridging molecule species in one molecule is particularly useful for the present invention. It is preferred to carry out Step (C) in the method of producing the substrate-protein complex as described in advance using this photobridable bridging molecule. Compounds that satisfy such condition will be illustrated below.

### Chemical formula (2): N-hydroxysuccinimide ester of 4-[3-(trifluoromethyl)-3H-diazirin-3-yl]benzoic acid,

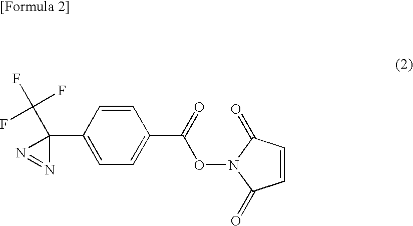

### Chemical formula (3): 4-[3-(trifluoromethyl)-3H-diazirin-3-yl]benzyl isothiocyanate

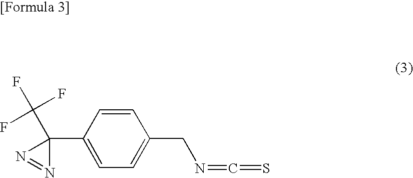

### Chemical formula (4): N-Hydroxysuccinimidyl-4-azidobenzoate

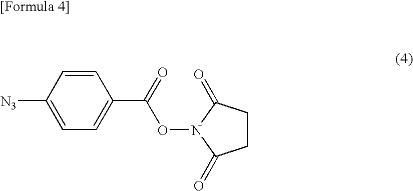

### Chemical formula (5): N-Hydroxysuccinimidyl-4-azidosalicylic acid,

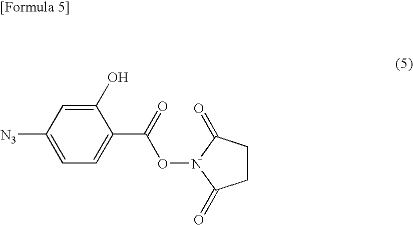

### Chemical formula (6): Sulfosuccinimidyl 2-(p-azidosalicylamido)ethyl-1-3′-dithiopropionate,

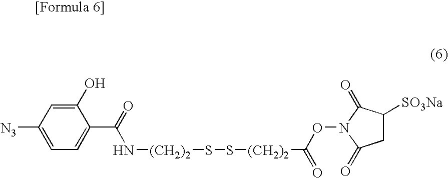

### Chemical formula (7): Sulfosuccinimidyl 4-(azidosalicylamido)hexanoate,

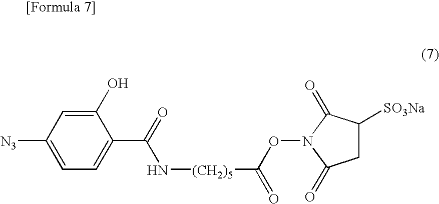

Moreover, divalent bridging agents such as compounds described below can also be used.
N-5-Azido-2-nitrobenzoyloxysuccinimide,
Sulfosuccinimidyl2-[m-azido-o-nitrobenzamido]-ethyl-1,3′-dithiopropionate,
(N-Succinimidyl-6-[4¹-azido-2′-nitrophenylamino]hexanoate,
Sulfosuccinimidyl-6-[4′-azido-2′-nitrophenylamino]hexanoate.

### <Target Substance>

The “target substance” according to the present invention used can be any molecule, as long as it may become an antigen by various techniques that employ antigen antibody reaction. In the “target substance capturing element,” “target substance detecting element” and “target substance separating element” of the present invention, the antigen may become the target substance.

The target substance of the present invention is broadly classified into non-biological substance or biological substance.

Examples of a non-biological substance of great industrial utility value include drug candidate molecules for various diseases related to for example obesity, diabetes and metabolic disease, neurological disease, psychotherapy, urogenital disease, reproduction and sexual medicine, inflammation, cancer, tissue repair, dermatology, chromatosis, photoaging, weakness, osteoporosis, cardiovascular disease, gastrointestinal disease, anti-infectivity, allergy and respiratory disease, sense organ disorder, sleeping disorder and hair loss; PCBs with different numbers/positions of chlorine substitution as environmental contaminants; dioxins with different numbers/positions of chlorine substitution as with PCBs; the so-called environmental hormones which are endocrine disruptors (such as hexachlorobenzene, pentachlorophenol, 2,4,5-trichloroacetic acid, 2,4-dichlorophenoxy acetic acid, amitrole, atrazine, alachlor, hexachlorocyclohexane, ethyl parathion, chlordane, oxychlordane, nonachlor, 1,2-dibromo-3-chloropropane, DDT, kelthane, aldrin, endrin, dieldrin, endosulfan (benzoepin), heptachlor, heptachlor epoxide, malathion, methomyl, methoxychlor, mirex, nitrofen, toxaphene, trifluralin, alkylphenol (5 to 9 carbon atoms), nonylphenol, octynylphenol, 4-octylphenol, bisphenol A, di-2-ethylhexyl phthalate, butylbenzyl phthalate, di-n-butyl phthalate, dicyclohexyl phthalate, diethyl phthalate, benzo(a)pyrene, 2,4-dichlorophenol, di-2-ethylhexyl adipate, benzophenone, 4-nitrotoluene, octachlorostyrene, aldicarb, benomyl, kepone (chlordecone), manzeb (mancozeb), maneb, metiram, metribuzin, cypermethrin, esfenvalerate, fenvalerate, permethrin, vinclozolin, zineb, ziram, dipentyl phthalate, dihexyl phthalate, dipropyl phthalate).

Examples of a biological substance include a biological substance selected from the group consisting of a nucleic acid, protein, sugar chain, lipid and a complex thereof. More specifically, the present invention can be applied to any substance as long as it contains a biomolecule selected from the group consisting of a nucleic acid, protein, sugar chain and lipid, and in particular as long as it contains a substance selected from any substance selected from the group consisting of a DNA, RNA, aptamer, gene, chromosome, cell membrane, virus, antigen, antibody, lectin, hapten, hormone, receptor, enzyme, peptide, sphingoglyco and sphingolipid. In addition, bacteria and cells themselves that produce the “biological substances” may also be the target substance as a “biological substance” intended by the present invention. Specific proteins include the so-called disease markers.

Examples of disease marker which can be mentioned are: α-fetoprotein (AFP) which is an acid glycoprotein produced in hepatocytes in fetal life and exists in the fetal blood, and can be a marker for hepatic cell carcinoma (primary liver cancer), hepatoblastoma, metastatic liver cancer and yolk sac tumor; PIVKA-II which is abnormal prothrombin appearing in hepatic parenchymal disorder and is confirmed to appear specifically in hepatic cell carcinoma; BCA225 which is immunohistochemically breast cancer specific glycoprotein and can be a marker for primary advanced breast cancer and recurrent and metastatic breast cancer; basic fetoprotein (BFP) which is basic fetal protein found in human fetal serum, intestine and brain tissue extract and is a marker for ovarian cancer, testis tumor, prostate cancer, pancreatic cancer, carcinoma of the biliary tract, hepatic cell carcinoma, kidney cancer, lung cancer, stomach cancer, bladder carcinoma and large bowel cancer; CA15-3 which is a carbohydrate antigen and can be a marker for advanced breast cancer, recurrent breast cancer, primary breast cancer and ovarian cancer; CA19-9 which is a carbohydrate antigen and can be a marker for pancreatic cancer, carcinoma of the biliary tract, stomach cancer, liver cancer, large bowel cancer and ovarian cancer; CA72-4 which is a carbohydrate antigen and can be a marker for ovarian cancer, breast cancer, colon and rectal cancer, stomach cancer and pancreatic cancer; CA125 which is a carbohydrate antigen and can be a marker for ovarian cancer (especially serous cystadenocarcinoma), adenocarcinoma of uterine body, cancer of the fallopian tubes, cervix adenocarcinoma, pancreatic cancer, lung cancer and large bowel cancer; CA130 which is a carbohydrate antigen and can be a marker for epithelial ovarian cancer, cancer of the fallopian tubes, lung cancer, hepatic cell carcinoma and pancreatic cancer; CA602 which is a core protein antigen and can be a marker for ovarian cancer (especially mucinous cystadenoma), adenocarcinoma of uterine body and cervix adenocarcinoma; CA54/61 (CA546) which is a mother carbohydrate associated antigen and can be a marker for ovarian cancer (especially mucinous cystadenoma), cervix adenocarcinoma and adenocarcinoma of uterine body; carcinoembryonic antigen (CEA) which is the most widely used at present for an aid in cancer diagnoses as a tumor-associated marker antigen of large bowel cancer, stomach cancer, rectal cancer, carcinoma of the biliary tract, pancreatic cancer, lung cancer, breast cancer, uterine cancer, urinary tract cancer, etc.; DUPAN-2 which is a carbohydrate antigen and can be a marker for pancreatic cancer, carcinoma of the biliary tract, hepatic cell carcinoma, stomach cancer, ovarian cancer and large bowel cancer; elastase 1 which exists in the pancreas and is a pancreatic exocrine proteinase specifically hydrolyzing elastic fiber elastin of the connective tissue (constituting the arterial wall, tendon, etc.) and can be a marker for pancreatic cancer, pancreatic cystic cancer and carcinoma of the biliary tract; immunosuppressive acidic protein (IAP) which is a glycoprotein found in high levels in ascites fluid and serum in human cancer patients and can be a marker for lung cancer, leukemia, esophageal cancer, pancreatic cancer, ovarian cancer, kidney cancer, bile duct adenocarcinoma, stomach cancer, bladder cancer, large bowel cancer, thyroid cancer and malignant lymphoma; NCC-ST-439 which is a carbohydrate antigen and can be a marker for pancreatic cancer, carcinoma of the biliary tract, breast cancer, large bowel cancer, hepatic cell carcinoma, pulmonary adenocarcinoma and stomach cancer; γ-seminoprotein (γ-Sm) which is a glycoprotein and can be a marker for prostatic cancer; prostate specific antigen (PSA) which is a glycoprotein extracted from human prostatic tissues and existing only in the prostatic tissue and accordingly can be a prostatic cancer marker; prostate acid phosphatase (PAP) which is excreted from the prostate and is an enzyme hydrolyzing phosphate ester under acidic pH and is used as tumor marker of prostate cancer; neuron specific enolase (NSE) which is the glycolytic pathway enzyme specifically existing in the nerve tissue and the neuroendocrine cell and can be a marker for lung cancer (especially lung small cell carcinoma), neuroblastomas, nerve system tumor, pancreatic islet tumor, esophageal small cell carcinoma, stomach cancer, kidney cancer and breast cancer; squamous cell carcinoma associated antigen (SCC antigen) which is a protein extracted and purified from liver metastatic focus of cervix squamous cell carcinoma and can be a marker for uterine cancer (cervix squamous cell carcinoma), lung cancer, esophageal cancer, head and neck tumor and skin cancer; sialyl Lex-I antigen (SLX) which is a carbohydrate antigen and can be a marker for pulmonary adenocarcinoma, esophageal cancer, stomach cancer, large bowel cancer, rectal cancer, pancreatic cancer, ovarian cancer and uterine cancer; SPan-1 which is a carbohydrate antigen and can be a marker for pancreatic cancer, carcinoma of the biliary tract, liver cancer, stomach cancer and large bowel cancer; tissue polypeptide antigen (TPA) which is a marker for esophageal cancer, stomach cancer, colorectal cancer, breast cancer, hepatic cell carcinoma, carcinoma of the biliary tract, pancreatic cancer, lung cancer and uterine cancer, and is a single chain polypeptide particularly useful for predicting advanced cancer by combining with other tumor maker and prognosis of recurrence and therapeutic process follow-up; sialyl Tn antigen (STN) which is a mother carbohydrate associated antigen and can be a marker for ovarian cancer, metastatic ovarian cancer, stomach cancer, large bowel cancer, biliary cancer, pancreatic cancer and lung cancer; CYFRA (cytokeratin) which is a tumor marker useful for detecting non small cell cancer of the lung, especially squamous cell carcinoma of the lung; pepsinogen (PG) which is inactive precursor of two type of pepsin (PG I and PG II), proteinase secreting in the gastric juice and can be a marker for gastric ulcer (especially low gastric ulcer), duodenal ulcer (especially recurrent and intractable cases), Brunner adenoma, Zollinger-Ellison syndrome and acute gastritis; C-reactive protein (CRP) which is an acute phase protein increased in plasma caused by tissue damage and infection and exhibits high level when myocardial necrosis is developed due to acute myocardial infarction; serum amyloid A (SAA) protein which is an acute phase protein increased in plasma caused by tissue damage and infection; myoglobin which is a heme protein having molecular weight about 17,500 existing mainly in the cardiac muscle and skeletal muscle and can be a marker for acute myocardial infarction, muscular dystrophy, polymyositis and dermatomyositis; creatinine kinase (CK) (three types of isozyme including CK-MM derived from skeletal muscle, CK-BB derived from brain and smooth muscle and CK-MB derived from cardiac muscle, bound CK with mitochondrial isozyme and immunoglobulin (macro CK)) which is an enzyme existing mainly in soluble fraction of skeletal muscle and cardiac muscle and releasing in the blood flow by cell damage and can be a marker for acute myocardial infarction, hypothyroidism, progressive muscular dystrophy and polymyositis; troponin T which forms troponin complex with troponin I and C on the thin filament of the striated muscle and is a protein having molecular weight 39,000 involving regulation of muscle contraction and can be a marker for rhabdomyolysis, cardiac myopathy, myocardial infarction and renal failure; and ventricular cardiac muscle myosin light chain I which is a protein in any cells of the skeletal muscle and cardiac muscle and can be a marker for acute myocardial infarction, muscular dystrophy and renal failure because of indicating damage or necrosis of the skeletal muscle and cardiac muscle as a result of showing increased assay level. Further, the target substance includes recently noticed stress makers such as chromogranin A, thioredoxin, 8-OHdG, cortisol, etc.

### <Target Substance Capturing Element>

By directing the specific molecule to which the domain constituting the second group of the substrate-protein complex described above binds to be the target substance, the target substance capturing element can be obtained.

### <Method and Device for Detecting the Target Substance>

By reacting a target substance capturing element having the above constitution with a sample, and detecting the state in which the target substance is captured by the target substance capturing element when the sample comprises the target substance, detection of the presence of the target substance within the sample will be possible. The detection of the state in which the target substance is captured by the target substance capturing element can be carried out by directly or indirectly measuring physical and/or chemical change of the target substance capturing element before and after the capture of the target substance. For example, a detection method that utilizes a label using a dye utilized in various assay fields and a method of measuring the optical change based on the capture of the target substance can be used. In addition, a target substance detection device can be constituted using at least a reaction device on which a region is formed for reacting the target substance capturing element and the sample and a detection means for detecting the state in which the target substance is captured by the target substance capturing element.

### <Method and Device for Capturing the Target Substance>

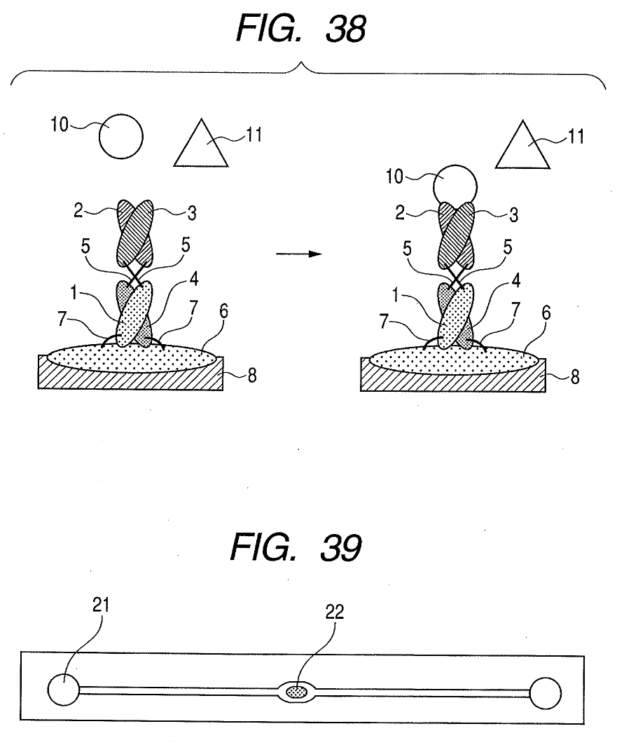

A target substance capturing element having the above constitution and the sample are reacted, and the target substance can be captured by the target substance capturing element when the sample comprises the target substance. By washing the target substance capturing element with which the target substance was captured, the target substance can be separated from other components comprised in the sample. FIG. 38 shows a schematic diagram of the step in which domains 2 and 3 of the target substance capturing element selectively captures a target substance 10 to which they may specifically bind to. The target substance 10 is distinguished from substance 11 that is not a subject of capture, and is captured by the target substance capturing element. In the target substance capturing element of the present invention, an inhibitor of non-specific adsorption or a derivative thereof that is adsorbed onto the surface of the substrate blocks the substrate surface, and the non-specific adsorption of the substance to the substrate can be effectively prevented. As a result, separation of the target substance with higher accuracy will be possible. In addition, a target substance capturing device can be constituted using at least a reaction device on which a region is formed for reacting the target substance capturing element and the sample and means for washing in the state where the target substance is captured by the target substance capturing element.

### <Applications of the Present Invention>

The binding protein molecule according to the present invention and the protein complex and substrate-protein complex using the same can be suitably utilized in a detection technique/kit/device etc. that utilize the molecular recognition action of the protein such as measurement by immunoassay, immunosensor and protein microarray. In addition, it can also be suitably utilized in a screening technique/kit/device, an immunochromatography technique/kit/device and a separation and purification technique/kit/device of chemical or biological substances etc. that utilizes the molecular recognition action of the protein.

### EXAMPLES

Effect of the present invention is illustrated concretely hereinbelow by reference to following examples. In the following examples, When egg white lysozyme (hereinafter sometimes designated as HEL) is used as an example of target substance. However, as described hereinabove, any substances can be used as the target substance in the present invention, if the corresponding antibody can be obtained and base and/or amino acid sequence of Fv region thereof is known.

### Example 1

### Obtainment of PEG- and HEL-Binding Bispecific Antibody Fragments

### (1) Preparation of PEG-Binding VH-Coding Nucleic Acid Fragment

An NcoI restriction site on the 5′-terminal side and an NheI restriction site on 3′-terminal side of the PEG-binding VH (SEQ ID NO: 49 or 50) disclosed in the description of WO 02/094853 were located. The PEG-binding VH for introducing vector (hereinafter designated as VH(PEG)) was prepared. Specifically, the overlapping PCR was performed by using following primers and applying a method according to the recommended protocol of the skilled artisan for the commercially available PCR kit to obtain artificial nucleic acids from base pairs of about 350 bp.

PEG-VH F-1

(SEQ ID NO: 13)

NNNNNCCATGGCAGGTCCAACTGCAGCAGCCCGGTGCTGAGCTTGTGAAG
CCTGGGGCCTCAGTGAAGCTGTCCTGCAAGGCTTCTGG
PEG-VH F-2

(SEQ ID NO: 14)

GAAGCAGAGGCCTGGACAAGGCCTTGAGTGGATTGGAAATTCTTATCCTG
GTAGTAGTAGTACTAACTACAATGAGAAGTTCAAGAGC
PEG-VH F-3

(SEQ ID NO: 15)

TAGACACATCCTCCAGTACAGCCTACATGCAGCTCAGCAGCCTGACATCT
GACGACTCTGCGGTCTTTTATTGTGCAAGATCGGGGCC
PEG-VH R-1

(SEQ ID NO: 16)

CCTTGTCCAGGCCTCTGCTTCACCCAGTTTATCCAATAATTGGTGAAAAT
GTAGCCAGAAGCCTTGCAGGACAGC
PEG-VH R-2

(SEQ ID NO: 17)

GGCTGTACTGGAGGATGTGTCTACAGTCAGTGTGGCCTTGCTCTTGAACT
TCTCATTGTA
PEG-VH R-3

(SEQ ID NO: 18)

NNNNNGCTAGCTGCAGAGACAGTGACCAGAGTCCCTTGGCCCCAGGAAGC
AAACCAGGCCGTCCCAGTTGGCCCCGATCTTGCACAATAAAAGACC

BIGDYE-PCR was performed by using PEG-VH F1 and PEG-VH R-1 using the commercially available sequencing kit and the reaction liquid composition. PCR cycling conditions were 96° C. for 3 min followed by 30 cycles of 94° C. for 1 min, 50° C. for 1 min and 68° C. for 4 min, and 4° C. It was confirmed that a fragment having a base sequence encoding the desired VH was obtained.

### (2) Preparation of PEG-Binding VL-Coding Nucleic Acid Fragments

An NheI restriction site and a nucleic acid encoding the linker (GGGGS) on the 5′-terminal side, and an SacII restriction site on the 3′-terminal side of the PEG-binding VL (SEQ ID NO: 51 or 52) disclosed in the description of WO 02/094853 were located. The BSA-binding VL for introducing vector (hereinafter designated as VL(PEG)) was prepared. Specifically, the nucleic acid fragments were obtained by the similar manner as in (1) except that the following primers were used, and were confirmed to have the desired base sequence of VL.

PEG-VL F-1

(SEQ ID NO: 19)

NNNNNGCTAGCGGTGGCGGTGGCTCTGATGTTTTGATGACCCAAACTCCA
CTCTCCCTGCCTGTCAGTCTTGGAGATCAAGCCTCCATCTCTTGCAGATC
TAGTCAGAGC
PEG-VL F-2

(SEQ ID NO: 20)

TTTAGATTGGTACCTGCAGAAACCAGGCCAGTCTCCAAAGCTCCTGATCT
ACAAAGTTTCCAACCGATTTTCTGG
PEG-VL F-3

(SEQ ID NO: 21)

CAGATTTCACACTCAAGATCACCAGAGTGGAGGCTGAGGATCTGGGAGTT
TATTACTGCTTTCAAGG
PEG-VL R-1

(SEQ ID NO: 22)

GTTTCTGCAGGTACCAATCTAAATAGGTGTTTCCATTACTATGTACAATG
CTCTGACTAGATCTGCAAGAGATGG
PEG-VL R-2

(SEQ ID NO: 23)

GGTGATCTTGAGTGTGAAATCTGTCCCTGATCCCCTGCCACTGAACCTGT
CTGGGACCCCAGAAAATCGGTTGGAAACTTTGT
PEG-VL R-3

(SEQ ID NO: 24)

NNNNNCCGCGGAGCCCGTTTCAGCTCCAGCTTGGTCCCAGCACCGAACGT
GAGCGGAACATGTGAACCTTGAAAGCAGTAATAAACTCCCAGA

### (3) Preparation of HEL-Binding VH-Coding Nucleic Acid Fragments

The HEL-binding VH (hereinafter designated as VH(HEL)) was prepared to incorporate it into a vector where an NcoI restriction site was located on the 5′-terminal side and an NheI restriction site on the 3′-terminal side in the HEL-binding VH (SEQ ID NO: 53 or 54). Specifically, the nucleic acid fragments were obtained by the same manner as in (1) except that the following primers were used and were confirmed to have the desired base sequence of VH.

HEL-VH F1

(SEQ ID NO: 25)

NNNNNCCATGGGATATCCAGCTGCAGGAGTCGGGCCCGAGCCTCGTCAAG
CCGTCGCAGACCCTGTCGCTCACCTGCAGCGTCACCGGCGA
HEL-VH F2

(SEQ ID NO: 26)

GCCTCGAGTACATGGGCTACGTCAGCTACTCGGGCAGCACCTACTACAAC
CCCTCGCTGAAGAGCCGCATCTCGATCACCCGCGACACCT
HEL-VH F3

(SEQ ID NO: 27)

GCCACCTACTACTGCGCGAACTGGGACGGCGACTACTGGGGCCAGGGCAC
CCTCGTCACCGTCTCCGCG
HEL-VH R1

(SEQ ID NO: 28)

GTAGCCCATGTACTCGAGGCGGTTGCCGGGGAACTTGCGGATCCACGACC
AGTAGTCCGAGGTGATCGAGTCGCCGGTGACGCTGCAGGT
HEL-VH R2

(SEQ ID NO: 29)

TTCGCGCAGTAGTAGGTGGCGGTGTCCTCGGTGGTGACCGAGTTGAGGTC
CAGGTAGTACTGGTTCTTGGAGGTGTCGCGGGTGATCGAG
HEL-VH R3

(SEQ ID NO: 30)

NNNNN GCTAGCCGCGGAGACGGTGACGAGG

### (4) Preparation of HEL-Binding VL-Coding Nucleic Acid Fragments

In the HEL-binding VL (Sequence D, SEQ ID NO: 55 or 56), an NheI restriction site and a nucleic acid encoding a linker (GGGGS) were located on the 5′-terminal side, and an SacII restriction site on the 3′-terminal side. The HEL-binding VL (hereinafter designated as VL(HEL)) was thus prepared to introduce it in a vector. Specifically, the nucleic acid fragments were obtained by the same manner as in (1) except that the following primers were used and were confirmed to have the desired base sequence of VL.

HEL-VL F1

(SEQ ID NO: 31)

NNNNNGCTAGCGGTGGCGGTGGCTCTGATATCGTCCTGACCCAGAGCCCG
GCGACCCTCTCGGTCACCCCCGGCAACTCGGTGTCCCTCTCGTGCCGCGC
CTCGCA
HEL-VL F2

(SEQ ID NO: 32)

CGCGCCTCCTGATCAAGTACGCCAGCCAGTCGATCTCGGGGATCCCGTCG
CGCTTCAGCGGCTCGGGCTCGGGCACCGAC
HEL-VL F3

(SEQ ID NO: 33)

GCATGTACTTCTGCCAGCAGTCGAACAGCTGGCCGTACACCTTCGGCGGC
GGTACCAAGCTGATCATCACGGCC
HEL-VL R1

(SEQ ID NO: 34)

GTACTTGATCAGGAGGCGCGGGCTCTCGTGCGACTTCTGCTGATACCAGT
GGAGGTTGTTGCCGATCGACTGCGAGGCGCGGCACGAGAG
HEL-VL R2

(SEQ ID NO: 35)

CTGCTGGCAGAAGTACATGCCGAAGTCCTCCGTCTCGACGCTGTTGATCG
ACAGGGTGAAGTCGGTGCCCGAGCCCGAGC
HEL-VL R3

(SEQ ID NO: 36)

NNNNN CCGCG G GGCCGTGATGATCAGCTTGGTACC

### (5) Preparation of Expression Vector

Two expression vectors were constructed in the following constitution by using above four types of nucleic acid fragments.

(5-1) Preparation of Vector (pPEG-HEL) for Expression of VH(PEG)-VL(HEL) (FIG. 41)

### (i) Insertion of VH(PEG)

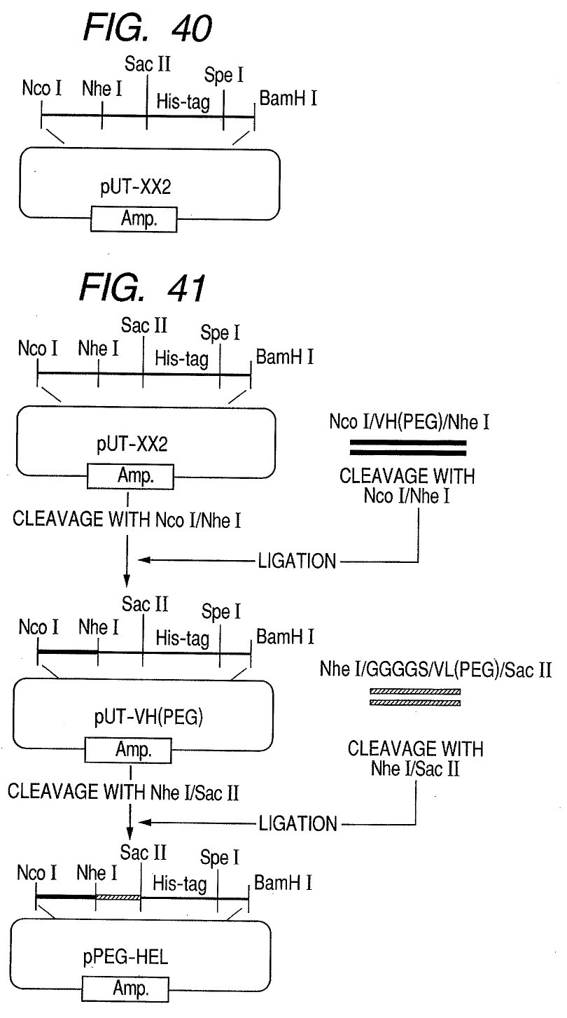

Plasmid pUT-XX2 shown in FIG. 40 was cleaved by restriction enzyme NcoI/NheI (New England Biolabs, Inc.) and treated with spin column S-400 HR (GE Health Care Biosciences). Similarly, the VHpeg, which was previously cleaved by NcoI/NheI, was cleaved and the cleavage fragment was purified by using the commercially available gel purification kit (SV Gel and PCR Clean-up system: Promega Corp.). Two fragments hereinabove were ligated by using the commercially available T4 ligase kit (Roche Inc.) according to the preparation protocol recommended by the maker. After transformation of JM109 competent cell 40 μl (Promega Corp.) with the ligation solution by heat shock, the transformed cells were plated on the LB/ampicillin (amp.) plate and incubated at 37° C. for overnight in static condition.

Subsequently, arbitrary colonies selected from the plate were inoculated into the LB/amp. liquid medium 3 ml and shake cultured at 37° C. for overnight. Thereafter, plasmid was collected by using the commercially available Mini Preps kit (Plus Minipreps DNA Purification System: Promega Corp.). DNA sequence of the obtained plasmid was confirmed according to the aforementioned sequencing method by using PEG-VH F1 and PEG-VH R1 to ensure insertion of the desired fragment.

### (ii) Insertion of VL(HEL)

Plasmid pUT-VH(PEG) obtained in the above (i) was cleaved by restriction enzyme NheI/SacII and treated with spin column S-400 HR (Amersham Biosciences Inc.). VL(PEG) cleaved by NheI/SacII was obtained by the same way. Ligation was performed by the same procedure as in the above (i), and the desired plasmid pPHEL for expression of VHpeg-VLh(VH(PEG)-VL(HEL)) was confirmed by the similar manner as in the above (i) (the primers for confirmation were VL-HEL F1 and VL-HEL R1).

### (5-2) Preparation of Vector (Phel-Peg) for Expression of VH(HEL)-VL(PEG)

### (i) Insertion of VH(HEL)

VHh was inserted into the plasmid pUT-XX2 by the same method as in above (5-1)(i), and the obtained plasmid was confirmed to be the desired plasmid (Primers for confirmation were HEL-VH F1 and HEL-VH R1).

### (ii) Insertion of VL(PEG)

VLpeg was inserted into the plasmid obtained in the above (i) by the same method as in the above (5-1)(ii), and the obtained plasmid was confirmed to be the desired vector pHEL-PEG for expression of VHh-VLpeg(VH(HEL)-VL(PEG)) by the similar manner as in the above (1) (the primers for confirmation were PEG-VL F1 and PEG-VL R1).

### (6) Protein Expression and Purification

Expression vectors which expressing polypeptides of VHpeg-VLh obtained in the above (5-1)(ii) and VHh-VLpeg obtained in (5-2)(ii) were treated in the protein expression process and the purification process in each independent system described hereinbelow to obtain polypeptide chains pPEG-HEL and pHEL-PEG.

### a) Transformation

BL21(DE3) competent cell solution 40 μL was transformed separately by each of the two expression vectors hereinabove. The transformation was performed by heat shock process including ice-cooling, 42° C. for 90 sec, and ice-cooling. LB medium 750 μL was added to the BL21 solution which was transformed by the heat shock and the mixture was shake cultured at 37° C. for 1 hour. Then the cultured liquid was centrifuged at 6,000 rpm for 5 min. The cultured supernatant 650 μL was discarded and the remained culture supernatant and precipitated cell fraction were stirred, plated on the LB/amp. plate and allowed to incubate statically at 37° C. for overnight.

### b) Preliminary Culture

Colonies on the plate were selected at random and shake cultured at 28° C. for overnight in the LB/amp. medium 3.0 ml.

### c) Main Culture

The above preliminary culture liquid was inoculated into 2×YT medium 750 ml, and further cultured at 28° C. When OD600 of the medium exceeded a value 0.8, IPTG was added to the final concentration at 1 mM, and the culture was continued at 28° C. for overnight.

### d) Purification

The desired polypeptide chain in the insoluble particle fraction was purified by the following process.

### (i) Recovery of Insoluble Particles

The culture liquid obtained in above c) was centrifuged at 6,000 rpm for 30 min. to obtain microbial cell fraction of the precipitate. The obtained microbial cells were suspended in Tris solution (20 mM Tris/500 mM NaCl) 15 ml in ice-cooling. The resultant cells in the suspension were disrupted with a French press to obtain cell disruption solution. The cell disruption solution was centrifuged at 12,000 rpm for 15 min., removed the supernatant and the precipitate was obtained as the insoluble particle fraction.

### (ii) Solubilization of Insoluble Particle Fraction

A 10 ml of 6 M guanidine hydrochloride/Tris solution was added to the insoluble fraction obtained in the above (i), and immersed overnight. The mixture was centrifuged at 12,000 rpm for 10 min. to obtain the supernatant as solubilized solution.

(iii) Metal Chelate Column

His-Bind (Novagen Corp.) was used as the metal chelate column carrier. Preparation of column, charging sample and washing procedure was performed at room temperature (20° C.) according to the manufacturer's recommendation. The desired His-Tagged polypeptide was eluted by using 60 mM imidazole/Tris solution. SDS-PAGE (acrylamide 15%) of the eluate showed single band, which confirmed the sample to be purified.

### (iv) Dialysis

The eluate hereinabove was dialyzed at 4° C. against 6 M guanidine hydrochloride/Tris solution to remove imidazole in the eluate to obtain each polypeptide chain solution hereinabove.

### (v) Refolding

Each polypeptide of VHpeg-VLh or VHh-VLpeg was refolded by dialysis at 4° C. comprising the following steps similar to the above dialysis and to remove guanidine hydrochloride.

(a) A 7.5 μM sample (volume after dilution: 10 ml) was prepared in 6 M guanidine hydrochloride/Tris solution from values of molar absorbance coefficient and ΔO.D. (280 nm-320 nm) of each polypeptide chain. β-mercaptoethanol (reducing agent) was added to the final concentration of 375 μM (fifty-fold concentration to the protein) and the reduction was performed at room temperature for 4 hours in the dark place. The sample solution was packed in the dialysis pack (MWCO: 14,000) to prepare sample for dialysis.
(b) The sample for dialysis was dialyzed for 6 hours gently with stirring against the external solution of 6 M guanidine hydrochloride/Tris solution.
(c) The concentration of guanidine hydrochloride in the external solution was reduced stepwisely to 3 M and 2 M. The sample was dialyzed against each external solution for 6 hours.
(d) To the Tris solution was added oxidized glutathione (GSSG) to the final concentration of 375 μM and L-Arg to the final concentration of 0.4 M. Further, 2 M external dialysis solution of the above (c) was added for adjusting the concentration of guanidine hydrochloride to 1 M. The sample was dialyzed at 4° C. for 12 hours with gently stirring against the 1 M guanidine solution with adjusting pH to 8.0 by adding NaOH.
(e) A Tris solution containing 0.5 M guanidine hydrochloride and L-Arg was prepared in a similar procedure to that in (d) and the sample was further dialyzed against the prepared Tris solution for 12 hours.
(f) Finally, the sample was dialyzed against Tris solution for 12 hours.
(g) After dialysis, the sample was centrifuged at 10,000 rpm for about 20 minutes to separate the aggregate and the supernatant.

### (vi) Purification of Dimeric Fragment

Each 5 μM polypeptide (VHpeg-VLh and VHh-VLpeg) solution obtained in the above (v) was mixed and allowed to stand at 4° C. for overnight. The mixed solution was passed through a Sephadex 75 column (column: 20 mM Tris buffer, 500 mM NaCl, flow rate: 1 ml/min.) to obtain a dimerized fraction corresponding to 60 kDa (about 18 minutes from injection).

(vii) Replacement of Buffer

The buffer was replaced by phosphate buffer (PBS) for the binding experiment hereinbelow. The replacement was carried out through dialysis. As dialysis pack, RC Membrane Spectra/Pro2 (MWCO: 12,000-14,000, Spectrum Chemical & Laboratory Products, Inc.) was used. Dimeric protein fraction obtained in (vi) packed in the dialysis pack was dialyzed gently with stirring against PBS 500 mL with three changes of the buffer solution every 6 hours. The thus obtained solution was used for sample for SPR measurement.

### Example 2

### Immobilization of Poly(Ethylene Glycol) (PEG) on Gold-Coated Substrate

Binding ability of the dimeric protein fraction obtained in example 1 on PEG adsorbed on the gold-coated substrate was measured by SPR. BIA core 2000 (BIAcore Inc.) was used as SPR measuring device. Unused SIA-kit Au gold-coated substrate was immersed in ethanol solution of PEG3-OH alkanethiol (Toyobo K. K. SPSPT0011) 10 mg/ml to form monolayer having PEG chain on the surface of gold-coated substrate. After washing the substrate with ethanol solution, the substrate was dried under nitrogen atmosphere. The gold-coated substrate was mounted on the SPR device and the running buffer was injected. After stabilizing SPR signal, 40 μl of 500 nM dimeric protein/PBST solution obtained in example 1 was injected. A curve indicating binding ability to PEG was obtained. Other experimental conditions are shown as follows.

Temperature: 25° C.

Flow rate: 1 μl/min.

### Example 3

### Evaluation of HEL-Binding Ability by SPR Measurement

Subsequent to the sample, which was evaluated the PEG-binding ability by SPR in example 2, 0.5, 1.0 and 2.0 μM HEL/PBST solution was injected consecutively. Binding ability of the dimeric protein fraction, which was bound with the immobilized PEG on the gold-coated substrate, to HEL was measured by SPR. SPR signal intensity shows dependent manner to the HEL concentration. Other experimental conditions are shown as follows.

Temperature: 25° C.

Flow rate: 1 μl/min.

### Example 4

### Obtainment of PC- and HEL-Binding Bispecific Antibody Fractions

### (1) Preparation of PC-Binding VH-Coding Nucleic Acid Fragment

In the PC-binding VH (Sequence x, SEQ ID NO: 57 or 58) disclosed in Protein Engineering, 12(7), 605-611 (1999) an NcoI restriction site was located on the 5′-terminal side and an NheI restriction site on the 3′-terminal side. The PEG-binding VH (hereinafter designated as VH(PC)) was thus prepared to introduce it in a vector. Specifically, the nucleic acid fragments were obtained by the same manner as in example 1 except that the following primers were used, and were confirmed to have the desired base sequence of VL.

PC-VH F1

(SEQ ID NO: 37)

NNNNNCCATGGGAAGTTAAATTAGTTGAATCTGGTGGTGGTTTAGTTCAA
CCTGGTGGTTCTTTACGTTTATCTTGTG
PC-VH F2

(SEQ ID NO: 38)

TGGAATGGGTTCGTCAACCTCCTGGTAAACGTTTAGAATGGATTGCTGCT
TCTCGTAATAAAGCTAATGATTATACTAC
PC-VH F3

(SEQ ID NO: 39)

GTGATACTTCTCAATCTATTTTATATTTACAAATGAATGCTTTACGTGCT
GAAGATACTGCTATTTATTATTGTGCTCG
PC-VH R1

(SEQ ID NO: 40)

AGGTTGACGAACCCATTCCATATAAAAATCAGAAAAAGTAAAACCAGAAG
TAGCACAAGATAAACGTAAAGAACC
PC-VH R2

(SEQ ID NO: 41)

TAAAATAGATTGAGAAGTATCACGAGAAACAATAAAACGACCTTTAACAG
AAGCAGAATATTCAGTAGTATAATCATTAGCTTTATTACG
PC-VH R3

(SEQ ID NO: 42)

NNNNNGCTAGCAGAAGAAGTAACAGTAGTACCAGCACCCCAAACATCAAA
ATACCAATAAGAAGAACCATAATAATCACGAGCACAATAATAAATAGC

### (2) Preparation of PC-Binding VL-Coding Nucleic Acid Fragments

In the PC-binding VL (SEQ ID NO: 59 or 60), an NheI restriction site and a nucleic acid encoding a linker (GGGGS) were located on the 5′-terminal side, and an SacII restriction site on the 3′-terminal side. The PC-binding VL (hereinafter designated as VL(PC)) was thus prepared to introduce it into a vector. Specifically, the nucleic acid fragments were obtained by the same manner as in (1) except that the following primers were used, and were confirmed to have the desired base sequence of VL.

PC-VL F1

(SEQ ID NO: 43)

NNNNNGCTAGCGGTGGCGGTGGCTCTGATATTGTTATGACTCAATCTCCT
ACTTTTTTAGCTGTTACTGCTTCTAAAAAAGTTACTATTTCTTGTACTGC
TTCTG
PC-VL F2

(SEQ ID NO: 44)

CATGTTCATTATTTAGCTTGGTATCAAAAAAAACCTGAACAATCTCCTAA
ATTATTAATTTATGGTGCTTCTAATCGTTATATTGG
PC-VL F3

(SEQ ID NO: 45)

CTGATTTTACTTTAACTATTTCTTCTGTTCAAGTTGAAGATTTAACTCAT
TATTATTGTG
PC-VL R1

(SEQ ID NO: 46)

AAGCTAAATAATGAACATGTTTAGAAGAATATAAAGATTCAGAAGCAGTA
CAAGAAATAGTAACTTTTTTAGAAGC
PC-VL R2

(SEQ ID NO: 47)

AATAGTTAAAGTAAAATCAGTACCAGAACCAGAACCAGTAAAACGATCAG
GAACACCAATATAACGATTAGAAGCACC
PC-VL R3

(SEQ ID NO: 48)

NNNNNCCGCGGACGTTTTAATTCTAATTTAGTACCAGCACCAAAAGTTAA
AGGATAAGAATAAAATTGAGCACAATAATAATGAGTTAAATC

### (5) Preparation of Expression Vector

Two expression vectors were constructed in the following constitution by using above four types of nucleic acid fragments ((1)HEL-binding VH-coding nucleic acid, (2) HEL-binding VL-coding nucleic acid fragment, (3) PC-binding VH-coding nucleic acid fragment and (4) PC biding VL-coding nucleic acid fragment) with similar manners as in example 1.

(5-1) Preparation of a Vector (pPC-HEL) for Expression of VH(PC)-VL(HEL)

### (i) Insertion of VH(PC)

VH(PC) was inserted into the plasmid pUT-XX2 by the similar manner as in example 1, (5-1)(i), and the obtained plasmid was confirmed to be the desired plasmid. (Primers for confirmation were VH(PC) F1 and VH(PC)R1.)

### (ii) Insertion of VL(HEL)

VL(HEL) was inserted into the plasmid obtained in (i) hereinabove by the same manner as in example 1, (5-1)(ii), and the obtained plasmid was confirmed by the similar manner as of (1) to be the desired plasmid, a vector pPC-HEL for expression of VH(PC)-VL(HEL). (Primers for confirmation were HEL-VL F1 and HEL-VL R1.)

(5-2) Preparation of Vector (pHEL-PC) for Expression of VH(HEL)-VL(PC)

### (i) Insertion of VH(HEL)

VH(HEL) was inserted into the plasmid pUT-XX2 by the similar manner as in (5-1)(i) hereinabove, and the obtained plasmid was confirmed to be an desired plasmid. (Primers for confirmation were HEL-VH F1 and HEL-VH R1.)

### (ii) Insertion of VL(PC)

VL(PC) was inserted into the plasmid obtained in the above (i) by the same method as in the above (5-1)(ii), and the obtained plasmid was confirmed to be the desired vector pHEL-PC for expression of VH(HEL)-VL(PC)) by the similar manner as in the above (1) (the primers for confirmation were HEL-VL F1 and HEL-VL R1).

### (6) Protein Expression and Purification

Expression vectors which expressing polypeptides of VH(PC)-VL(HEL) obtained in the above (5-1)(ii) and VH(HEL)-VL(PC) obtained in (5-2)(ii) were treated by the similar manner as in example 1(6) to obtain dimeric proteins of VH(PC)-VL(HEL) and VH(HEL)-VL(PC).

### Example 5

### Immobilization of Phosphatidyl Choline (PC) on Gold-Coated Substrate

Binding ability of the dimeric protein fraction obtained in example 4 on PC adsorbed on the gold-coated substrate was measured by SPR with the similar manner as in example 2. PC was immobilized on a surface of the gold thin film by using unused SPR sensor tip HPA. 10 mM chloroform solution of phosphatidyl choline (Sigma Inc.) was prepared as PC. The solution was dried under reduced pressure in an evaporator, and dried further with a vacuum pump for 2 hours. After drying, PBS 0.5 ml was added to prepare suspension. Freezing and thawing were repeated 5 times with spraying liquid nitrogen. Liposome was prepared by using liposome preparation equipment (AVESTIN Inc.). After washing the flow path system of the SPR device, PC was immobilized by using liposome solution adjusted to 0.5 mM with HBS-N buffer (Biacore International AB). A curve indicating binding ability to PC was obtained. Other experimental conditions are shown as follows.

Temperature: 25° C.

Flow rate: 5 μl/min.

Surface active agent (40 mM n-Octyl-β-glucoside): 5 min

Flow rate: 5 μl/min.

Liposome solution: 30 min.

50 mM NaOH: 1 min.

Running buffer (PBST)

### Example 6

### Evaluation of HEL-Binding Ability by SPR Measurement

Subsequent to the sample, which was evaluated the PC-binding ability by SPR in example 5, 0.5, 1.0 and 2.0 μM HEL/PBST solution was injected consecutively. Binding ability of the dimeric protein fraction, which was bound with the immobilized PC on the gold-coated substrate, to HEL was measured by SPR. SPR signal intensity shows dependent manner to the HEL concentration. Other experimental conditions are shown as follows.

Temperature: 25° C.

Flow rate: 1 μl/min.

### Example 7

### Evaluation of PEG Bridging Binding Protein Molecule

Following experimental operations were performed at 4° C. in the light shielding condition. Surface of a gold-coated substrate of the unused SIA-Kit Au gold-coated substrate was immersed in 10 mg/ml ethanol solution of PEG6-COOH alkanethiol. Acetate buffer solution of 0.2 M N(3-dimethylaminopropyl)-N′-ethylcarbodiimide (EDC)/0.05M N-hydroxysuccinimide ester of 4-[3-(trifluoromethyl)-3H-diazirine-3-yl]benzoic acid was added and reacted for 24 hours. After washing the substrate with water, the substrate was irradiated with the light of the high-pressure mercury vapor lamp from 330 nm to 400 nm for 1 hour under nitrogen atmosphere with maintaining at 15° C. The substrate prepared by the procedure as above was mounted on an SPR device similar to that used in example 1. Running buffer was run through the flow path to wash out superfluous sample. 1 μM HEL solution was injected by the similar manner as in example 3, and biding ability of sample of photobridging protein adsorbed on the gold-coated substrate to HEL was measured by means of SPR. SPR signal intensity shows dependent manner to the HEL concentration. Other experimental conditions are shown as follows.

Temperature: 25° C.

Flow rate: 1 μl/min.

### Example 8

### Preparation of Element for Detection of Target Substance Using Peg Bridging Binding Protein Molecule and Evaluation Thereof

A micro-fluid tip shown in FIG. 39 was manufactured by molding PDMS. A flow path without upper cover was at first filled with terminal aminated PEG alkanethiol/ethanol solution, and after allowing to stand for 30 minutes, the flow path was washed with ethanol. After purging the flow path with nitrogen, acetate buffer was spotted on a position 22 indicated in FIG. 39 by using liquid dispenser (SHOTMASTER 300, Musashi Engineering K. K.) at 4° C. under light shielding. Acetate buffer contained 4-[3-(trifluoromethyl)-3H-diazirine-3-yl]benzoic acid N-hydroxysuccinimide ester. In order to avoid evaporation of the solution, the tip was put a cover on, allowed to stand, and a reaction between NHS group of ester and amino group of PEG terminal was performed. After 24 hours of the reaction, the flow path was washed to purge nitrogen, then the binding protein molecule solution prepared in example 1 was spotted on the same spotting position, and irradiated with the light of the high-pressure mercury vapor lamp from 330 nm to 400 nm for 1 hour with maintaining at 15° C. After washing with PBST solution, the cover was immobilized. 1 μM HEL solution was introduced with the flow rate 2 μl/min. from the introducing port 21 in FIG. 39. After rewashing with PBST solution, the flow path was filled with PBST solution of 1 μM anti-HEL antibody, and allowed to stand one hour later, the flow path was washed three times with PBST solution, filled with PBST solution of 10 μM rhodamine bound anti-IgG antibody and allowed to stand. One hour later, the flow path was washed three times with PBST solution, and was observed by fluorescent microscope. As a result, fluorescence was observed on the position where the binding protein molecule hereinbefore was spotted. Subsequently, when concentration of target substance HEL solution was diluted to 1/10 and 1/100, and the same operation was performed, the fluorescent intensity was confirmed to correlate with the concentration of HEL.

This application claims priority from Japanese Patent Application No. 2005-253337 filed on Sep. 1, 2005, which is hereby incorporated by reference herein.

1. A binding protein molecule, characterized in that it has at least one component selected from a first group consisting of components (1) and (4) and at least one component selected from a second group consisting of components (2) and (3), wherein components (1) to (4) are described as follows:
(1) A domain having a binding site to an inhibitor of non-specific adsorption, wherein the domain comprises at least a part of the variable region of an antibody heavy chain,
(2) A domain having a binding site to a target substance, wherein the domain comprises at least a part of the variable region of an antibody light chain,
(3) A domain having a binding site to the target substance, wherein the domain comprises at least a part of the variable region of an antibody heavy chain, and
(4) A domain having a binding site to the inhibitor of non-specific adsorption, wherein the domain comprises at least a part of the variable region of an antibody light chain.

2. A binding protein molecule, characterized in that it has a first domain having a binding site to the inhibitor of non-specific adsorption in which the domain comprises a part of the variable region of an antibody as the binding site and a second domain having a binding site to the target substance in which the domain comprises a part of the variable region of an antibody as the binding site, wherein the first and second domains are bound via a linker.

3. The binding protein molecule according to claim 1 having a linked body in which two of the components are linked.

4. The binding protein molecule according to claim 3, wherein the linked body is linked in combinations of said components (1) and (2) and components (3) and (4).

5. The binding protein molecule according to claim 3, wherein the linkage of said linked body comprises a peptide structure comprising one or more amino acids.

6. The binding protein molecule according to claim 1, wherein the inhibitor of non-specific adsorption is a polymer comprising in at least a portion thereof a functional group capable of inhibiting non-specific adsorption.

7. The binding protein molecule according to claim 6, wherein the polymer comprising in at least a portion thereof the functional group capable of inhibiting non-specific adsorption is a polymer comprising in at least a portion thereof an ethylene glycol group.

8. The binding protein molecule according to claim 7, wherein the polymer comprising in at least a portion thereof an ethylene glycol group is at least one polymer selected from the group consisting of polyethylene glycol and derivatives thereof.

9. The binding protein molecule according to claim 8, wherein the domain of the component (1) comprises one or more amino acid sequences as shown in SEQ ID NO: 1 to SEQ ID NO: 3 below:
SEQ ID NO: 1: NYWIN,
SEQ ID NO: 2: NSYPGSSSTNYNEKFK
and
SEQ ID NO: 3: YCARSG.

10. The binding protein molecule according to claim 8, wherein the amino acid sequence of the domain of the component (1) has one or more amino acid sequences selected from the group consisting of (A) to (F) below:
(A) The amino acid sequence of SEQ ID NO: 1 (NYWIN),
(B) The amino acid sequence of SEQ ID NO: 2 (NSYPGSSSTNYNEKFK),
(C) The amino acid sequence of SEQ ID NO: 3 (YCARSG),
(D) An amino acid sequence formed by providing deletion, substitution or addition one or several amino acids into the amino acid sequence of SEQ ID NO: 1,
(E) An amino acid sequence formed by providing deletion, substitution or addition one or several amino acids into the amino acid sequence of SEQ ID NO: 2, and
(F) An amino acid sequence formed by providing deletion, substitution or addition one or several amino acids into the amino acid sequence of SEQ ID NO: 3.

11. The binding protein molecule according to claim 8, wherein the domain of the component (4) comprises one or more amino acid sequences as shown in SEQ ID NO: 4 to SEQ ID NO: 6 below:
SEQ ID NO: 4: RSSQSIVHSNGNTYLD,
SEQ ID NO: 5: KVSNRFS
and
SEQ ID NO: 6: FQGSHVPLT.

12. The binding protein molecule according to claim 8, wherein the amino acid sequence of the domain of the component (4) has one or more amino acid sequences selected from the group consisting of (A) to (F) below:
(A) The amino acid sequence of SEQ ID NO: 4 (RSSQSIVHSNGNTYLD),
(B) The amino acid sequence of SEQ ID NO: 5 (KVSNRFS),
(C) The amino acid sequence of SEQ ID NO: 6 (FQGSHVPLT),
(D) An amino acid sequence formed by providing deletion, substitution or addition one or several amino acids into the amino acid sequence of SEQ ID NO: 4,
(E) An amino acid sequence formed by providing deletion, substitution or addition one or several amino acids into the amino acid sequence of SEQ ID NO: 5, and
(F) An amino acid sequence formed by providing deletion, substitution or addition one or several amino acids into the amino acid sequence of SEQ ID NO: 6.

13. The binding protein molecule according to claim 6, wherein the polymer comprising in at least a portion thereof the functional group capable of inhibiting non-specific adsorption is a polymer comprising in at least a portion thereof a phospholipid group.

14. The binding protein molecule according to claim 13, wherein the molecule comprising in at least a portion thereof a phospholipid group is a polymer comprising in a portion thereof at least one group selected from the group consisting of a phosphatidyl choline group and derivatives thereof.

15. The binding protein molecule according to claim 14, wherein the domain of the component (1) comprises one or more amino acid sequences as shown in SEQ ID NO: 7 to SEQ ID NO: 9 below:
SEQ ID NO: 7: GFTFSDFYME,
SEQ ID NO: 8: ASRNKANDYTTEYSASVK
and
SEQ ID NO: 9: DYYGSSYWYFDV.

16. The binding protein molecule according to claim 14, wherein the amino acid sequence of the domain of the component (1) has one or more amino acid sequences selected from the group consisting of (A) to (F) below:
(A) The amino acid sequence of SEQ ID NO: 7 (GFTFSDFYME),
(B) The amino acid sequence of SEQ ID NO: 8 (ASRNKANDYTTEYSASVK),
(C) The amino acid sequence of SEQ ID NO: 9 (DYYGSSYWYFDV),
(D) An amino acid sequence formed by providing deletion, substitution or addition one or several amino acids into the amino acid sequence of SEQ ID NO: 7,
(E) An amino acid sequence formed by providing deletion, substitution or addition one or several amino acids into the amino acid sequence of SEQ ID NO: 8, and
(F) An amino acid sequence formed by providing deletion, substitution or addition one or several amino acids into the amino acid sequence of SEQ ID NO: 9.

17. The binding protein molecule according to claim 14, wherein the domain of the component (4) comprises one or more amino acid sequences as shown in SEQ ID NO: 10 to SEQ ID NO: 12 below:
SEQ ID NO: 10: TASESLYSSKHVHYLA;
SEQ ID NO: 11: GASNRYI
and
SEQ ID NO: 12: AQFYSYPL.

18. The binding protein molecule according to claim 14, wherein the amino acid sequence of the domain of the component (4) has one or more amino acid sequences selected from the group consisting of (A) to (F) below:
(A) The amino acid sequence of SEQ ID NO: 10 (TASESLYSSKHVHYLA),
(B) The amino acid sequence of SEQ ID NO: 11 (GASNRYI),
(C) The amino acid sequence of SEQ ID NO: 12 (AQFYSYPL),
(D) An amino acid sequence formed by providing deletion, substitution or addition one or several amino acids into the amino acid sequence of SEQ ID NO: 10,
(E) An amino acid sequence formed by providing deletion, substitution or addition one or several amino acids into the amino acid sequence of SEQ ID NO: 11, and
(F) An amino acid sequence formed by providing deletion, substitution or addition one or several amino acids into the amino acid sequence of SEQ ID NO: 12.

19. A protein complex having a binding protein molecule with a binding site to a target substance and an inhibitor of non-specific adsorption bound to the binding protein molecule, characterized in that the binding protein molecule with a binding site to the target substance is the binding protein molecule according to claim 1.

20. A substrate-protein complex having a substrate, an inhibitor of non-specific adsorption disposed on at least a portion of the substrate and a binding protein molecule with a binding site to a target substance, characterized in that the binding protein molecule with a binding site to the target substance is the binding protein molecule according to claim 1.

21. A method of producing a substrate-protein complex having a substrate and a protein with a binding site to a target substance, characterized in that it has the steps of:
(step A) disposing an inhibitor of non-specific adsorption on a surface of the substrate; and
(step B) binding the inhibitor of non-specific adsorption disposed on the substrate to the protein with a binding site to the target substance,
wherein the protein with a binding site to the target substance is the binding protein molecule according to claim 1.

22. The production method according to claim 21, which in addition to the (step A) and (step B) comprises the step of:
(step C) forming a covalent bond between the inhibitor of non-specific adsorption and the binding protein molecule via a bridging molecule.

23. The production method according to claim 22, wherein the bridging molecule comprises a photoreactive atomic group.

24. A target substance capturing element having a substrate and a target substance capturing protein immobilized to a surface of the substrate, in which a region where the target substance capturing protein is immobilized is a reactive region, characterized in that the target substance capturing protein is the protein molecule according to claim 1 and the protein molecule is immobilized to the substrate surface via an inhibitor of non-specific adsorption disposed on the reactive region.

25. A method of detecting a target substance, characterized in that it has the steps of:
contacting the target substance capturing element according to claim 24 with a sample; and
detecting a state in which the target substance is captured by the target substance capturing element when the sample contains the target substance.

26. A detection device for a target substance, characterized in that it has:
a reactive region for reacting the target substance capturing element according to claim 24 with a sample; and
a detection means for detecting a state in which the target substance is captured by the target substance capturing element.

27. A method of capturing a target substance, characterized in that it has the steps of:
contacting the target substance capturing element according to claim 24 with a sample to capture the target substance by the target substance capturing element; and
washing the target substance capturing element by which the target substance has been captured to separate the captured target substance from the substance that has not been captured by the target substance capturing element.

28. A capturing device for a target substance, characterized in that it has:
a reactive region for reacting a target substance capturing element according to claim 24 with a sample; and
means for washing and removing the target substance captured by the target substance capturing element and the substance that has not been captured by the target substance capturing element from the target substance capturing element.


SEQUENCE LISTING (60 sequences)

SEQ ID NO: 1 - protein, 5 aa, Artificial
  Domain for binding to polyethylene glycol
NYWIN

SEQ ID NO: 2 - protein, 16 aa, Artificial
  Domain for binding to polyethylene glycol
NSYPGSSSTNYNEKFK

SEQ ID NO: 3 - protein, 6 aa, Artificial
  Domain for binding to polyethylene glycol
YCARSG

SEQ ID NO: 4 - protein, 16 aa, Artificial
  Domain for binding to polyethylene glycol
RSSQSIVHSNGNTYLD

SEQ ID NO: 5 - protein, 7 aa, Artificial
  Domain for binding to polyethylene glycol
KVSNRFS

SEQ ID NO: 6 - protein, 9 aa, Artificial
  Domain for binding to polyethylene glycol
FQGSHVPLT

SEQ ID NO: 7 - protein, 10 aa, Artificial
  Domain for binding to phosphorylcholine
GFTFSDFYME

SEQ ID NO: 8 - protein, 18 aa, Artificial
  Domain for binding to phosphorylcholine
ASRNKANDYTTEYSASVK

SEQ ID NO: 9 - protein, 12 aa, Artificial
  Domain for binding to phosphorylcholine
DYYGSSYWYFDV

SEQ ID NO: 10 - protein, 16 aa, Artificial
  Domain for binding to phosphorylcholine
TASESLYSSKHVHYLA

SEQ ID NO: 11 - protein, 7 aa, Artificial
  Domain for binding to phosphorylcholine
GASNRYI

SEQ ID NO: 12 - protein, 8 aa, Artificial
  Domain for binding to phosphorylcholine
AQFYSYPL

SEQ ID NO: 13 - DNA, 88 bp, Artificial
  primer
nnnnnccatggcaggtccaactgcagcagcccggtgctgagcttgtgaagcctggggcctcagtgaagctgtcctgcaaggcttctgg

SEQ ID NO: 14 - DNA, 88 bp, Artificial
  Primer
gaagcagaggcctggacaaggccttgagtggattggaaattcttatcctggtagtagtagtactaactacaatgagaagttcaagagc

SEQ ID NO: 15 - DNA, 88 bp, Artificial
  Primer
tagacacatcctccagtacagcctacatgcagctcagcagcctgacatctgacgactctgcggtcttttattgtgcaagatcggggcc

SEQ ID NO: 16 - DNA, 75 bp, Artificial
  Primer
ccttgtccaggcctctgcttcacccagtttatccaataattggtgaaaatgtagccagaagccttgcaggacagc

SEQ ID NO: 17 - DNA, 60 bp, Artificial
  Primer
ggctgtactggaggatgtgtctacagtcagtgtggccttgctcttgaacttctcattgta

SEQ ID NO: 18 - DNA, 96 bp, Artificial
  Primer
nnnnngctagctgcagagacagtgaccagagtcccttggccccaggaagcaaaccaggccgtcccagttggccccgatcttgcacaataaaagacc

SEQ ID NO: 19 - DNA, 110 bp, Artificial
  Primer
nnnnngctagcggtggcggtggctctgatgttttgatgacccaaactccactctccctgcctgtcagtcttggagatcaagcctccatctcttgcagatctagtcagagc

SEQ ID NO: 20 - DNA, 75 bp, Artificial
  Primer
tttagattggtacctgcagaaaccaggccagtctccaaagctcctgatctacaaagtttccaaccgattttctgg

SEQ ID NO: 21 - DNA, 67 bp, Artificial
  Primer
cagatttcacactcaagatcaccagagtggaggctgaggatctgggagtttattactgctttcaagg

SEQ ID NO: 22 - DNA, 75 bp, Artificial
  Primer
gtttctgcaggtaccaatctaaataggtgtttccattactatgtacaatgctctgactagatctgcaagagatgg

SEQ ID NO: 23 - DNA, 83 bp, Artificial
  Primer
ggtgatcttgagtgtgaaatctgtccctgatcccctgccactgaacctgtctgggaccccagaaaatcggttggaaactttgt

SEQ ID NO: 24 - DNA, 93 bp, Artificial
  Primer
nnnnnccgcggagcccgtttcagctccagcttggtcccagcaccgaacgtgagcggaacatgtgaaccttgaaagcagtaataaactcccaga

SEQ ID NO: 25 - DNA, 91 bp, Artificial
  Primer
nnnnnccatgggatatccagctgcaggagtcgggcccgagcctcgtcaagccgtcgcagaccctgtcgctcacctgcagcgtcaccggcga

SEQ ID NO: 26 - DNA, 90 bp, Artificial
  Primer
gcctcgagtacatgggctacgtcagctactcgggcagcacctactacaacccctcgctgaagagccgcatctcgatcacccgcgacacct

SEQ ID NO: 27 - DNA, 69 bp, Artificial
  Primer
gccacctactactgcgcgaactgggacggcgactactggggccagggcaccctcgtcaccgtctccgcg

SEQ ID NO: 28 - DNA, 90 bp, Artificial
  Primer
gtagcccatgtactcgaggcggttgccggggaacttgcggatccacgaccagtagtccgaggtgatcgagtcgccggtgacgctgcaggt

SEQ ID NO: 29 - DNA, 90 bp, Artificial
  Primer
ttcgcgcagtagtaggtggcggtgtcctcggtggtgaccgagttgaggtccaggtagtactggttcttggaggtgtcgcgggtgatcgag

SEQ ID NO: 30 - DNA, 30 bp, Artificial
  Primer
nnnnngctagccgcggagacggtgacgagg

SEQ ID NO: 31 - DNA, 106 bp, Artificial
  Primer
nnnnngctagcggtggcggtggctctgatatcgtcctgacccagagcccggcgaccctctcggtcacccccggcaactcggtgtccctctcgtgccgcgcctcgca

SEQ ID NO: 32 - DNA, 80 bp, Artificial
  Primer
cgcgcctcctgatcaagtacgccagccagtcgatctcggggatcccgtcgcgcttcagcggctcgggctcgggcaccgac

SEQ ID NO: 33 - DNA, 74 bp, Artificial
  Primer
gcatgtacttctgccagcagtcgaacagctggccgtacaccttcggcggcggtaccaagctgatcatcacggcc

SEQ ID NO: 34 - DNA, 90 bp, Artificial
  Primer
gtacttgatcaggaggcgcgggctctcgtgcgacttctgctgataccagtggaggttgttgccgatcgactgcgaggcgcggcacgagag

SEQ ID NO: 35 - DNA, 80 bp, Artificial
  Primer
ctgctggcagaagtacatgccgaagtcctccgtctcgacgctgttgatcgacagggtgaagtcggtgcccgagcccgagc

SEQ ID NO: 36 - DNA, 35 bp, Artificial
  Primer
nnnnnccgcggggccgtgatgatcagcttggtacc

SEQ ID NO: 37 - DNA, 78 bp, Artificial
  Primer
nnnnnccatgggaagttaaattagttgaatctggtggtggtttagttcaacctggtggttctttacgtttatcttgtg

SEQ ID NO: 38 - DNA, 79 bp, Artificial
  Primer
tggaatgggttcgtcaacctcctggtaaacgtttagaatggattgctgcttctcgtaataaagctaatgattatactac

SEQ ID NO: 39 - DNA, 79 bp, Artificial
  Primer
gtgatacttctcaatctattttatatttacaaatgaatgctttacgtgctgaagatactgctatttattattgtgctcg

SEQ ID NO: 40 - DNA, 75 bp, Artificial
  Primer
aggttgacgaacccattccatataaaaatcagaaaaagtaaaaccagaagtagcacaagataaacgtaaagaacc

SEQ ID NO: 41 - DNA, 90 bp, Artificial
  Primer
taaaatagattgagaagtatcacgagaaacaataaaacgacctttaacagaagcagaatattcagtagtataatcattagctttattacg

SEQ ID NO: 42 - DNA, 98 bp, Artificial
  Primer
nnnnngctagcagaagaagtaacagtagtaccagcaccccaaacatcaaaataccaataagaagaaccataataatcacgagcacaataataaatagc

SEQ ID NO: 43 - DNA, 105 bp, Artificial
  Primer
nnnnngctagcggtggcggtggctctgatattgttatgactcaatctcctacttttttagctgttactgcttctaaaaaagttactatttcttgtactgcttctg

SEQ ID NO: 44 - DNA, 86 bp, Artificial
  Primer
catgttcattatttagcttggtatcaaaaaaaacctgaacaatctcctaaattattaatttatggtgcttctaatcgttatattgg

SEQ ID NO: 45 - DNA, 60 bp, Artificial
  Primer
ctgattttactttaactatttcttctgttcaagttgaagatttaactcattattattgtg

SEQ ID NO: 46 - DNA, 76 bp, Artificial
  Primer
aagctaaataatgaacatgtttagaagaatataaagattcagaagcagtacaagaaatagtaacttttttagaagc

SEQ ID NO: 47 - DNA, 78 bp, Artificial
  Primer
aatagttaaagtaaaatcagtaccagaaccagaaccagtaaaacgatcaggaacaccaatataacgattagaagcacc

SEQ ID NO: 48 - DNA, 92 bp, Artificial
  Primer
nnnnnccgcggacgttttaattctaatttagtaccagcaccaaaagttaaaggataagaataaaattgagcacaataataatgagttaaatc

SEQ ID NO: 49 - DNA, 360 bp, Artificial
  Anti-PEG heavy chain variable region
caggtccaactgcagcagcccggtgctgagcttgtgaagcctggggcctcagtgaagctgtcctgcaaggcttctggctacattttcaccaattattggataaactgggtgaagcagaggcctggacaaggccttgagtggattggaaattcttatcctggtagtagtagtactaactacaatgagaagttcaagagcaaggccacactgactgtagacacatcctccagtacagcctacatgcagctcagcagcctgacatctgacgactctgcggtcttttattgtgcaagatcggggccaactgggacggcctggtttgcttcctggggccaagggactctggtcactgtctctgca

SEQ ID NO: 50 - protein, 120 aa, Artificial
  Anti-PEG heavy chain variable region
QVQLQQPGAELVKPGASVKLSCKASGYIFTNYWINWVKQRPGQGLEWIGNSYPGSSSTNYNEKFKSKATLTVDTSSSTAYMQLSSLTSDDSAVFYCARSGPTGTAWFASWGQGTLVTVSA

SEQ ID NO: 51 - DNA, 342 bp, Artificial
  Anti-PEG light chain variable region
gatgttttgatgacccaaactccactctccctgcctgtcagtcttggagatcaagcctccatctcttgcagatctagtcagagcattgtacatagtaatggaaacacctatttagattggtacctgcagaaaccaggccagtctccaaagctcctgatctacaaagtttccaaccgattttctggggtcccagacaggttcagtggcaggggatcagggacagatttcacactcaagatcaccagagtggaggctgaggatctgggagtttattactgctttcaaggttcacatgttccgctcacgttcggtgctgggaccaagctggagctgaaacgggct

SEQ ID NO: 52 - protein, 114 aa, Artificial
  Anti-PEG light chain variable region
DVLMTQTPLSLPVSLGDQASISCRSSQSIVHSNGNTYLDWYLQKPGQSPKLLIYKVSNRFSGVPDRFSGRGSGTDFTLKITRVEAEDLGVYYCFQGSHVPLTFGAGTKLELKRA

SEQ ID NO: 53 - DNA, 339 bp, Artificial
  Anti-HEL heavy chain variable region
gatatccagctgcaggagtcgggcccgagcctcgtcaagccgtcgcagaccctgtcgctcacctgcagcgtcaccggcgactcgatcacctcggactactggtcgtggatccgcaagttccccggcaaccgcctcgagtacatgggctacgtcagctactcgggcagcacctactacaacccctcgctgaagagccgcatctcgatcacccgcgacacctccaagaaccagtactacctggacctcaactcggtcaccaccgaggacaccgccacctactactgcgcgaactgggacggcgactactggggccagggcaccctcgtcaccgtctccgcg

SEQ ID NO: 54 - protein, 113 aa, Artificial
  Anti-HEL heavy chain variable region
DVQLQESGPSLVKPSQTLSLTCSVTGDSTTSDYWSWIRKFPGNRLEYMGYVSYSGSTYYNPSLKSRISITRDTSKNQYYLDLNSVTTEDTATYYCANWDGDYWGQGTLVTVSA

SEQ ID NO: 55 - DNA, 324 bp, Artificial
  Anti-HEL light chain variable region
gatatcgtcctgacccagagcccggcgaccctctcggtcacccccggcaactcggtgtccctctcgtgccgcgcctcgcagtcgatcggcaacaacctccactggtatcagcagaagtcgcacgagagcccgcgcctcctgatcaagtacgccagccagtcgatctcggggatcccgtcgcgcttcagcggctcgggctcgggcaccgacttcaccctgtcgatcaacagcgtcgagacggaggacttcggcatgtacttctgccagcagtcgaacagctggccgtacaccttcggcggcggtaccaagctgatcatcacggcc

SEQ ID NO: 56 - protein, 108 aa, Artificial
  Anti-HEL light chain variable region
DIVLTQSPATLSVTPGNSVSLSCRASQSIGNNLHWYQQKSHESPRLLTKYASQSISGTPSRFSGSGSGTDFTLSINSVETEDFGMYFCQQSNSWPYTFGGGTKLEITA

SEQ ID NO: 57 - DNA, 366 bp, Artificial
  Anti-PC heavy chain variable region
gaagttaaattagttgaatctggtggtggtttagttcaacctggtggttctttacgtttatcttgtgctacttctggttttactttttctgatttttatatggaatgggttcgtcaacctcctggtaaacgtttagaatggattgctgcttctcgtaataaagctaatgattatactactgaatattctgcttctgttaaaggtcgttttattgtttctcgtgatacttctcaatctattttatatttacaaatgaatgctttacgtgctgaagatactgctatttattattgtgctcgtgattattatggttcttcttattggtattttgatgtttggggtgctggtactactgttacttcttct

SEQ ID NO: 58 - protein, 122 aa, Artificial
  Anti-PC heavy chain variable region
EVKLVESGGGLVQPGGSLRLSCATSGFTFSDFYMEWVRQPPGKRLEWIAASRNKANDYTTEYSASVKGRFIVSRDTSQSILYLQMNALRAEDTAIYYCARDYYGSSYWYFDVWGAGTTVTSS

SEQ ID NO: 59 - DNA, 339 bp, Artificial
  Anti-PC light chain variable region
gatattgttatgactcaatctcctacttttttagctgttactgcttctaaaaaagttactatttcttgtactgcttctgaatctttatattcttctaaacatgttcattatttagcttggtatcaaaaaaaacctgaacaatctcctaaattattaatttatggtgcttctaatcgttatattggtgttcctgatcgttttactggttctggttctggtactgattttactttaactatttcttctgttcaagttgaagatttaactcattattattgtgctcaattttattcttatcctttaacttttggtgctggtactaaattagaattaaaacgt

SEQ ID NO: 60 - protein, 113 aa, Artificial
  Anti-PC light chain variable region
DIVMTQSPTFLAVTASKKVTISCTASESLYSSKNVHYLAWYQKKPEQSPKLLIYGASNRYIGVPDRFTGSGSGTDFTLTISSVQVEDLTHYYCAQFYSYPLTFGAGTKLELKR